(19) United States
(12) Patent Application Publication
(10) Pub. No.: US 2023/0003743 A1
(43) Pub. Date: Jan. 5, 2023

(54) METHODS AND COMPOSITIONS FOR TREATING OR PREVENTING AN ALLERGY OR ANAPHYLAXIS

(71) Applicant: THE CHILDREN'S MEDICAL CENTER CORPORATION, Boston, MA (US)

(72) Inventors: Talal Amine Chatila, Belmont, MA (US); Elena Crestani, Brookline, MA (US); Emmanuel Stephen Victor, Karnataka (IN); Azza Abdel-Gadir, Cambridge, MA (US); Rima Rachid, Belmont, MA (US)

(73) Assignee: THE CHILDREN'S MEDICAL CENTER CORPORATION, Boston, MA (US)

(21) Appl. No.: 17/801,238

(22) PCT Filed: Feb. 23, 2021
(86) PCT No.: PCT/US2021/019215

§ 371 Date: Aug. 19, 2022

Related U.S. Application Data
(60) Provisional application No. 62/980,778, filed on Feb. 24, 2020.

(51) Int. Cl. G01N 33/68; G01N 33/74; A61P 37/08

(52) U.S. Cl. CPC G01N 33/6893; G01N 33/74; A61P 37/08; G01N 2333/575; G01N 2800/24; G01N 2800/50

(57) ABSTRACT

Described herein are methods and compositions for treating or preventing an allergy or anaphylaxis. Certain aspects of the invention relate to administering to a subject an agent that inhibits RELMβ.

### CROSS REFERENCE TO RELATED APPLICATION

This application is a 35 U.S.C. § 371 National Phase Entry Application of International Patent Application No. PCT/US2021/019215 filed Feb. 23, 2021, which designated the U.S., which claims the benefit under 35 U.S.C. § 119(e) of U.S. Provisional Application No. 62/980,778, filed on Feb. 24, 2020, the contents of which are incorporated herein by reference in their entireties.

### GOVERNMENT SUPPORT

This invention was made with Government support under Grant Nos AI126915, AI117983, and AI132843 awarded by the National Institutes of Health. The Government has certain rights in the invention.

### FIELD OF THE INVENTION

The field of the invention relates to the treatment or prevention of an allergy or anaphylaxis.

### SEQUENCE LISTING

The instant application contains a Sequence Listing which has been submitted electronically in ASCII format and is hereby incorporated by reference in its entirety. Said ASCII copy, created on Mar. 30, 2021, is named 701039-094190WOPT_SL.txt and is 75,926 bytes in size.

### BACKGROUND

Food allergies (FA) have emerged as a prominent pediatric health issue in westernized countries, with 6-8% of children currently living with FA in the US alone. FA significantly affect the day-to-day life of pediatric patients as they involve all common food staples and are responsible for potentially life-threatening reactions upon accidental exposures. Yet, the understanding of mechanisms involved in FA pathogenesis—beyond the role of IgE—remains limited. This knowledge gap is immediately reflected in the challenges routinely faced by clinicians in identifying individuals at risk for FA, in providing accurate diagnosis and prognosis, and, finally, in precisely establishing when clinical tolerance is acquired over time. FA diagnosis currently relies on skin prick testing and/or specific IgE measurement. These IgE-based methodologies may not reflect clinical disease in the absence of a clear history of reactions and are limited in their ability to predict the acquisition of future tolerance. The current recommendation for FA subjects is strict allergen avoidance, which impacts quality of life and, importantly, does not promote the acquisition of tolerance over time. Further, this approach does not aid in the prevention and/or treatment of a severe reaction to a food allergy, for example, anaphylaxis.

More accurate biomarkers of FA are strongly needed to identify individuals at risk of developing FA, to assist in the accurate diagnosis and monitoring of subjects with FA, and to predict those individuals who are likely to have outgrown their FA. Such biomarkers will also be helpful in predicting and/or identifying a subject at risk of having a severe reaction, such as anaphylaxis, to an allergy, e.g., a food allergy. The need is also strong for more specific therapeutic interventions that alter the course of the disease and help imparting long-term tolerance.

### SUMMARY

This invention is based, in part, on the finding that subjects having a food allergy, but not asthmatics or non-allergic controls, have increased concentrations of Resistin-like beta (RELMβ) in their sera. RELMβ is also increased in the sera and gut tissues of FA-prone Il4ra^{F709} mice as compared to non FA-prone wild-type mice. Furthermore, RELMβ-deficient Il4ra^{F709} mice (Retnlb^{-/-}Il4ra^{F709} mice) are protected from anaphylaxis when sensitized and orally challenged with food allergens. RELMβ-deficiency also increased the frequencies of ROR-γt⁺ regulatory T (Treg) cells, an immune cell population critical for establishing oral immune tolerance to foods.

Accordingly, one aspect described herein provides a method for identifying a subject at risk of having anaphylaxis, the method comprising: (a) obtaining a biological sample from a subject; (b) measuring the level of Resistin-like beta (RELMβ) in the biological sample of (a); (c) comparing the level of (b) with a reference level, wherein a subject is identified as being at risk for anaphylaxis if the level of (b) is greater than a reference level; and, optionally, (d) administering to the subject identified as being at risk for anaphylaxis an anti-anaphylaxis therapeutic.

In one embodiment of any aspect provided herein, the anti-anaphylaxis therapeutic is an agent that inhibits RELMβ. In one embodiment of any aspect provided herein, the anti-anaphylaxis therapeutic is a microbiota therapeutic.

In one embodiment of any aspect provided herein, the method further comprises, prior to obtaining the biological sample, diagnosing a subject as having, or likely to develop, an allergy.

In one embodiment of any aspect provided herein, the method further comprises, prior to obtaining the biological sample, receiving the results of an assay that diagnoses a subject as having, or likely to develop, an allergy.

In one embodiment of any aspect provided herein, the subject is selected from the group consisting of: a newborn, an infant, a toddler, a child, and an adult.

In one embodiment of any aspect provided herein, the allergy is a food allergy. Exemplary allergies, e.g., that may also result in anaphylaxis, include a drug allergy, an insect allergy, a latex allergy, a mold allergy, a pet allergy, and a pollen allergy. Exemplary food allergies include soy, wheat, eggs, dairy, peanuts, tree nuts, shellfish, fish, mushrooms, stone fruits, and other fruits. In one embodiment of any aspect, the subject is allergic to one or more food allergens.

In one embodiment of any aspect provided herein, the agent is selected from the group consisting of: a small molecule, a compound, an antibody, a peptide, and an expression vector encoding an inhibitory nucleic acid or polypeptide. In one embodiment of any aspect provided herein, the antibody or antibody reagent is a humanized antibody or antibody reagent.

In one embodiment of any aspect provided herein, the vector is non-integrative or integrative. Exemplary non-integrative vectors include, but are not limited to, an episomal vector, an EBNA1 vector, a minicircle vector, a non-integrative adenovirus, a non-integrative RNA, and a Sendai virus. In one embodiment of any aspect provided herein, the vector is a lentivirus vector.

In one embodiment of any aspect provided herein, the agent increases the population of RORγt⁺ regulatory T cells. For example, the population of RORγt⁺ regulatory T cells is increased by at least 50%, 60%, 70%, 80%, 90%, 95%, 99%, or more as compared to the population of RORγt⁺ regulatory T cells prior to administration.

In one embodiment of any aspect provided herein, the agent reduces the level of RELMβ by at least 50%, 60%, 70%, 80%, 90%, 95% or more as compared to the level of RELMβ prior to administration. In one embodiment of any aspect provided herein, the expression of RELMβ is increased by at least 2×, 3×, 4×, 5×, 6×, 7×, 8×, 9×, or more as compared to the reference level. In one embodiment of any aspect provided herein, the reference level is the RELMβ level in a healthy patient.

In one embodiment of any aspect provided herein, the microbiota therapeutic is a fecal matter transplant and the fecal matter is obtained from a healthy subject.

In one embodiment of any aspect provided herein, the biological sample is a sera or tissue sample.

One aspect described herein provides a method for treating or preventing the onset of anaphylaxis in a subject, the method comprising: administering an agent that inhibits RELMβ to a subject.

One aspect described herein provides a method for inducing tolerance to an allergen in a subject, the method comprising: administering an agent that inhibits RELMβ to a subject.

One aspect described herein provides a method for reducing or eliminating a subject's immune reaction to an allergen, the method comprising: administering an agent that inhibits RELMβ to a subject.

In one embodiment of any aspect provided herein, the method further comprises, prior to administration, diagnosing a subject as having, or likely to develop, an allergy.

In one embodiment of any aspect provided herein, the method further comprises, prior to administration, receiving the results of an assay that diagnoses a subject as having, or likely to develop, an allergy.

In one embodiment of any aspect provided herein, the method further comprises, prior to administration, diagnosing a subject as having increased level of RELMβ as compared to the reference level.

In one embodiment of any aspect provided herein, the method further comprises, prior to administration, receiving the results of an assay that diagnoses a subject as having increased level of RELMβ as compared to the reference level.

One aspect described herein provides a composition comprising an agent that inhibits RELMβ.

one embodiment of any aspect provided herein, the composition further comprises a pharmaceutically acceptable carrier.

One aspect described herein provides a pharmaceutical composition comprising an agent that inhibits RELMβ.

One aspect described herein provides a use of any composition described herein for the prevention of anaphylaxis in subject having or at risk of developing an allergy.

One aspect described herein provides a use of any composition described herein for the treatment of anaphylaxis in subject having or at risk of developing an allergy.

One aspect described herein provides a use of any composition described herein for inducing tolerance to an allergen in subject having or at risk of developing an allergy.

One aspect described herein provides a use of any composition described herein for reducing or eliminating a subject's immune reaction to an allergen.

One aspect described herein provides a method for identifying a subject at risk of having anaphylaxis, the method comprising (a) obtaining a biological sample from a subject; (b) measuring the level of Resistin-like beta (RELMβ) in the biological sample of (a); and (c) comparing the level of (b) with a reference level, wherein a subject is identified as being at risk for anaphylaxis if the level of (b) is greater than a reference level.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with food allergies. The term “treating” includes reducing or alleviating at least one adverse effect or symptom of a food allergy (e.g., itching, gastric distress, or inflamed or constricted airway). Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein “preventing” or “prevention” refers to any methodology where the disease state or disorder (e.g., an allergen, such as a food allergy, or anaphylaxis) does not occur due to the actions of the methodology (such as, for example, administration of an anti-anaphylaxis agent, such as a RELMβ inhibitor, that increase the population of RORγt-expressing regulatory T cells, or a composition thereof. In one aspect, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease encompasses a reduction in the likelihood that a subject will develop the disease, relative to an untreated subject (e.g. a subject who is not treated with a composition comprising an agent as described herein).

As used herein, the term “allergen” refers to any naturally occurring protein, any modified protein, any recombinant protein, any recombinant mutant protein, or any protein fragment thereof or mixtures of proteins that have been reported to induce allergic reaction, i.e. IgE mediated reactions upon their repeated exposure to an individual.

Examples of naturally occurring allergens include pollen allergens (tree, weed, herb and grass pollen allergens), mite allergens (from e.g. house dust mites and storage mites), insect allergens (inhalant, saliva- and venom origin allergens), animal allergens from e.g. saliva, hair and dander from e.g. dog, cat, horse, rat, mouse, etc., fungi allergens and food allergens.

Important pollen allergens from trees, grasses and herbs are such originating from the taxonomic orders of Fagales, Oleales, Pinales and platanaceae including i.a. birch (Betula), alder (Alnus), hazel (Corylus), hornbeam (Carpinus), olive (Olea), cedar (Cryptomeria and Juniperus), Plane tree (Platanus), the order of Poales including i.a. grasses of the genera Lolium, Phleum, Poa, Cynodon, Dactylis, Holcus, Phalaris, Secale, and Sorghum and the orders of Asterales and Urticales including i.a. herbs of the genera Ambrosia, Artemisia, and Parietaria. Other important inhalation allergens are those from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (superfamily Apidae), wasps (superfamily Vespidea), and ants (superfamily Formicoidae). Important inhalation allergens from fungi are i.a. such originating from the genera Alternaria, Cladosporium, Aspergillus and Penicillium.

Examples of food allergens are allergens from wheat, crustacean food including shrimp, prawn, crab and lobster, fish, hen's eggs, peanut, soy, cows' milk, nuts such as almond, brazil nut, cashew nut, hazelnut and walnut, celery, mustard and sesame seed.

Examples of recombinant allergens include but are not limited to proteins/peptides from plant pollens, grass pollens, tree pollens, weed pollens, insect venom, dust and storage mite proteins, animal dander, saliva, fungal spores and food allergens (i.e., peanut, milk, gluten and egg) prepared using recombinant techniques. Recombinant allergens can be obtained e.g. on a large scale by using microbial expression systems that may be grown on fermenters, produced by recombinant DNA techniques, or chemical precursors or other chemicals when synthesized chemically.

As used herein, the term “food allergy” refers to a failure of oral tolerance to food antigens associated with Th2 immunity and allergen-specific IgE responses. That is, an immune response is generated in response to particular food antigens and can lead to hives, gastrointestinal symptoms, abdominal pain, anaphylaxis and even death.

As used herein, the term “tolerance” refers to the level of allergic response to a particular quantity of allergen.

As used herein, the term “antigen” refers to a substance or substances alone or in combination that when introduced into the lymphatic system induces production of antibodies that bind to a fraction of the molecule or molecules.

As used herein, the term “allergic reaction” refers to any untoward response to an allergen.

As used herein, the term “administering,” refers to the placement of a therapeutic (e.g., an agent that inhibits RELMβ) or pharmaceutical composition as disclosed herein into a subject by a method or route which results in at least partial delivery of the agent to the subject. Pharmaceutical compositions comprising agents as disclosed herein can be administered by any appropriate route which results in an effective treatment in the subject.

As used herein, “transplanting” refers to the placement of fecal matter, e.g. from a healthy subject, as described herein into a subject, by a method or route which results in at least partial localization of the introduced fecal matter at a desired site, such as the intestines or a region thereof, such that a desired effect(s) is produced (e.g., tolerance to a food allergen). The fecal matter can be administered by any appropriate route which results in delivery to a desired location in the subject where at least a portion of the delivered fecal matter remain viable. The period of viability of the fecal matter after administration to a subject can be as short as a few hours, e.g., twenty-four hours, to a few days, to as long as several years, i.e., long-term engraftment. In some embodiments, the fecal matter transplant is administered in the form of a suppository, pill, capsule, or the like. In some embodiments, the fecal matter transplant is administered orally, rectally, or enterically. In some embodiments, the fecal matter transplant is administered in the form of a liquid or solution. In some embodiments, the fecal matter transplant is administered using a colonoscopy, enema, or a plastic tube inserted through the nose into the gastrointestinal tract (e.g., stomach or intestines).

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., an allergy or anaphylaxis. A subject can be male or female. A subject can be a newborn (e.g., birth to 2 months), an infant (e.g., 2 months to 1 year), a toddler (e.g., 1 year to 4 year), a child (e.g., less than 18 years of age), or an adult (e.g., greater than 18 years of age).

A subject can be one who has been previously diagnosed with or identified as suffering from or having a disease or disorder in need of treatment (e.g., an allergy or anaphylaxis) or one or more complications related to such a disease or disorder, and optionally, have already undergone treatment for the disease or disorder or the one or more complications related to the disease or disorder. Alternatively, a subject can also be one who has not been previously diagnosed as having such disease or disorder (e.g., an allergy or anaphylaxis) or related complications. For example, a subject can be one who exhibits one or more risk factors for the disease or disorder or one or more complications related to the disease or disorder or a subject who does not exhibit risk factors. A “subject in need” of treatment for a particular condition can be a subject having that condition, diagnosed as having that condition, or at risk of developing that condition.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein, “microbiota therapeutic” refers to a therapeutic comprising, consisting of, or consisting essential of a beneficial microbial cell or by product, e.g., a non-pathogenic microbial cell, that is capable of increasing the population of RORγt-expressing Tregs after administration. The beneficial microbial cell to be administered can be live, dead, e.g., in a preserved state, such as dried. The microbial cell need not be viable to be beneficial. The bacterial cell can be administered as an intact, whole cell, or in pieces. Exemplary microbiota therapeutics include, but are not limited to a fecal matter transplant, a probiotic, a characterized microbial consortium, a single microbial strain or species, an engineered microbial strain or species, and a by-product of the microbial cell, such as a metabolite, e.g., indoxyl 3 sulfate (I3S), e.g., as described in PCT/US2019/060431, the contents of which is incorporated herein by reference in its entirety.

As used herein, “fecal matter transplant” refers to a transfer of stool from a healthy donor, e.g., a donor not having or at risk of having a food allergy, to a gastrointestinal tract of a subject. Previous terms for the procedure include fecal bacteriotherapy, fecal transfusion, fecal transplant, stool transplant, fecal enema, and human probiotic infusion (HPI). Because the procedure involves the complete restoration of the entire fecal microbiota, not just a single agent or combination of agents, these terms have now been replaced by the new term fecal microbiota transplantation. Methods for performing a fecal matter transplant are known in the art, for example, performed by colonoscopy and less commonly by nasoduodenal tube. During colonoscopy, the colonoscope is advanced through the entire colon. As the colonoscope is withdrawn, the fecal matter obtained from the healthy donor is delivered through the colonoscopy into the subject's colon. Fecal matter samples can be prepared and administered in various forms, for example, a freeze dried sample, a fresh sample, a blended sample, or a diluted sample. Methods for preparation and administration of a fecal matter sample are further described in, e.g., U.S. Pat. Nos. 9,192,361, 9,308,226, and 9,968,638; and International Patent Application No. WO2014152484 which are incorporated herein, in their entirety.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein, the terms “protein” and “polypeptide” are used interchangeably herein to refer to a polymer of amino acids. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wild-type reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g., nucleic acid or polypeptide which is differentially present in a sample taken from subjects having a food allergy, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to an appropriate control.

The terms “increase”, “enhance”, or “activate” are all used herein to mean an increase by a reproducible statistically significant amount. In some embodiments, the terms “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible statistically significant increase in such level.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with an allergen, e.g., a food allergy, or anaphylaxis, or a biological sample that has not been contacted with an agent disclosed herein).

As used herein, an “appropriate control” refers to an untreated, otherwise identical cell, population, or healthy subject (e.g., a patient or cell that was not administered an agent described herein, as compared to a patient with an allergy or anaphylaxis or a cell treated with an agent as described herein).

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIG. 1 shows the role of the metabolome in of the −omics spectrum.

FIGS. 2A-2D show dysregulation of RELMβ and RELMα in FA-prone Il4ra^{F709} mice. FIGS. 2A and 2B, qPCR analysis of Retnlb or Retnla transcripts in jejunal tissues of WT and Il4ra^{F709} mice that were either sham sensitized (PBS) or orally sensitized with OVA+ staphylococcal enterotoxin B (SEB), as described [43]. FIGS. 2B and 2C, Serum levels of RELMβ and RELMα in WT and Il4ra^{F709} mice pre- (FIG. 2C) and post-sensitization (FIG. 2D) with OVA/SEB, as determined by ELISA. N=4-6/group for FIG. 2A and 5-15/group for FIG. 2B and FIG. 2C. *p<0.05, **p<0.01 by one-way ANOVA and post-test analysis.

FIG. 3 shows RELMβ levels are selectively elevated in FA. Distribution of serum RELMβ concentrations in age matched healthy controls (n=10), asthmatics (n=26), asthmatics with FA (FAA) (n=20) and FA children (n=24). *p<0.05, **p<0.01, and ****p<0.0001 by one-way ANOVA and posttest (Kruskal Wallis) analysis.

FIGS. 4A-4E show Retnlb deficiency abrogates FA in Il4ra^{F709} mice. FIG. 4A, Core body temperature changes in Il4ra^{F709} and Il4ra^{F709} Retnlb^{-/-} mice that have been either sham (PBS) or OVA/SEB sensitized, as indicated, and then challenged with OVAFIG. FIG. 4B, Total and OVA-specific serum IgE concentrations in sham and OVA/SEB sensitized mice. FIG. 4C, serum MMCP-1 concentrations post OVA challenge. FIG. 4D, Toluidine blue staining of jejunal mast cells (indicated by arrows). FIG. 4E, mast cell counts per low powered field. *p<0.05 **p<0.01, ***p<0.001, ****p<0.0001 by one-way ANOVA with post-test analysis.

FIG. 5A, principal component analysis of untargeted metabolomic profiles of WT, Il4ra^{F709} single mutant and Il4ra^{F709} Retnlb^{-/-} double mutant mice by gender. FIG. 5B, Heatmap of selected pathways and metabolites significantly different (p<0.05) in Il4ra^{F709} Retnlb^{-/-} double mutant mice (R−/F709) as compared to WT and/or single mutant Il4ra^{F709} (F709) mice. Triangles indicate significant fold change<1, stars indicate significant fold change>1.

FIG. 6 shows heat-map of metabolites significantly different (p<0.005) between FA children and non-atopic controls. For each metabolite, a colorimetric representation of relative expression in each sample is shown according to the scale depicted on top. Metabolites are grouped into main dysregulated pathways and a miscellaneous category.

FIGS. 7A and 7B show small intestinal ROR-γt⁺ Treg cell deficiency in FA-prone Il4ra^{F709} mice is restored in Retnlb deficient mice. FIG. 7A, Flow cytometric analysis of gut ROR-γt⁺ Treg cells in OVA/SEB-sensitized WT,Il4ra^{F709} and Il4ra^{F709} Retnlb^{-/-} mice. Only the Il4ra^{F709} mice, but not the WT or Il4ra^{F709} Retnlb^{-/-} mice, developed anaphylaxis upon OVA challenge. FIG. 7B, Scatter plot representation of the frequency of ROR-γt⁺ Treg cells in the respective mouse groups. **P<0.01, ***P<0.001 by one-way ANOVA with post-test analysis.

### DETAILED DESCRIPTION

It is demonstrated herein, for the first time, that Resistin-like beta (RELMβ) is a biomarker for food allergy (FA) and is mechanistically involved in the disease process. FA subjects, but not asthmatics or non-allergic controls, have increased concentrations of RELMβ in their sera. RELMβ is also increased in the sera and gut tissues of FA-prone Il4raF709 mice as compared to non FA-prone wild-type mice. Furthermore, RELMβ-deficient Il4raF709 mice (Retnlb^{-/-}Il4raF709 mice) are protected from anaphylaxis when sensitized and orally challenged with food allergens. RELMβ-deficiency also increased the frequencies of ROR-γt+ regulatory T (Treg) cells, an immune cell population critical for establishing oral immune tolerance to foods. Data presented herein establish the feasibility of targeting the RELMβ pathway in the prevention of anaphylaxis in a subject having an allergy, and the boosting of oral immune tolerance to an allergen.

Anaphylaxis is a severe, potentially life-threatening allergic reaction. It can occur within seconds to minutes of exposure to an allergen that a subject is allergic to, such as a food allergy or bee sting.

Anaphylaxis causes immune system to release a flood of chemicals, which induces shock. The release of chemicals is triggered by an interaction between an allergic antibody (IgE) and the allergen. Common symptoms of anaphylaxis include, but are not limited to, a rapid drop in blood pressure (hypotension), constricted airway, a swollen tongue, wheezing and difficulty breathing, rapid and weak pulse, a skin reaction (such as hives and itching and flushed or pale skin), nausea and vomiting, dizziness or fainting, and death. Allergens that commonly trigger anaphylaxis include certain foods, some medications, insect venom and latex. Symptoms are commonly apparent within seconds to minutes of exposure to the allergen, and less commonly, within hours of exposure to the allergen. Up to 20 percent of subjects have a second wave of symptoms hours or days after their initial symptoms have subsided; this is called biphasic anaphylaxis.

One skilled in the art can diagnose a subject as having anaphylaxis by determining if the subject has elevated levels of the enzyme, tryptase, in a blood sample taken from the subject. Tryptase is elevated in the blood for at least 3 hours following anaphylaxis. Current treatments for anaphylaxis include, but are not limited to, Epinephrine (adrenaline, EpiPen®), supplemental oxygen, intravenous (IV) antihistamines and cortisone, and a beta-agonist (such as albuterol). There are currently no know methods for accurately predicting the severity of anaphylaxis, i.e., would the anaphylaxis episode result in mild symptoms or death. Previous reactions to an allergen is not a reliable predictor as to whether a subject with develop anaphylaxis, or the severity of an anaphylaxis episode.

### Treating or Preventing Anaphylaxis

Provided herein is a method for identifying a subject at risk of having anaphylaxis comprising: (a) obtaining a biological sample from a subject; (b) measuring the level of Resistin-like beta (RELMβ) in the biological sample of (a); (c) comparing the level of (b) with a reference level, wherein a subject is identified as being at risk for anaphylaxis if the level of (b) is greater than a reference level; and, optionally, (d) administering to the subject identified as being at risk for anaphylaxis an anti-anaphylaxis therapeutic.

In one embodiment, the level of RELMβ is increased by at least 10%, at least 20%, at least 30%, at least 40%, at least 50%, at least 60%, at least 70%, at least 80%, at least 90%, at least 95%, at least 99% or more, or at least 2×, 3×, 4×, 5×, 6×, 7×, 8×, 9×, or more as compared to the reference level as compared to the reference level. As used herein, the “reference level” refers to the level of RELMβ in a healthy subject, e.g., not having an allergy and/or at risk of having anaphylaxis. One skilled in the art can assess the mRNA or protein level of RELMβ, e.g., in a biological sample, using PCR-based assays or Westernblotting, respectively.

In one embodiment, the anti-anaphylaxis agent is an agent that inhibits RELMβ levels, for example, by at least 10%, at least 20%, at least 30%, at least 40%, at least 50%, at least 60%, at least 70%, at least 80%, at least 90%, at least 95%, at least 99%, or more as compared to a reference level. As used herein, “reference level” refers to the RELMβ levels prior to administration of the agent that inhibits RELMβ levels, or the RELMβ level in a subject having an allergy and/or anaphylaxis, or at risk of having allergy and/or anaphylaxis. One skilled in the art can assess the mRNA or protein level of RELMβ, e.g., in a biological sample, using PCR-based assays or Westernblotting, respectively.

In one embodiment, the anti-anaphylaxis agent is a microbial therapeutic, for example, a fecal matter transplant, wherein the fecal matter is obtained from a healthy subject. In one embodiment, the agent that inhibits RELMβ is co-administered with a microbial therapeutic.

In one embodiment, the method further comprises, prior to obtaining the biological sample, diagnosing a subject as having, or likely to develop, an allergy, i.e., a reaction to an allergen. In one embodiment, the method further comprises, prior to obtaining the biological sample, receiving the results of an assay that diagnoses a subject as having, or likely to develop, an allergy, i.e., a reaction to an allergen. Methods and assays for diagnosing a food allergy include, but are not limited to a complete family history of allergic disease, a blood test, for example, ImmunoCAP test), and/or a skin prick food allergy test that indicates if a subject has food-specific IgE antibodies. These assays are commonly known in the art and can be executed by a skilled clinician.

Examples of naturally occurring allergens include pollen allergens (tree, weed, herb and grass pollen allergens), mite allergens (from e.g. house dust mites and storage mites), insect allergens (inhalant, saliva- and venom origin allergens), animal allergens from e.g. saliva, hair and dander from e.g. dog, cat, horse, rat, mouse, etc., fungi allergens and food allergens.

Important pollen allergens from trees, grasses and herbs are such originating from the taxonomic orders of Fagales, Oleales, Pinales and platanaceae including i.a. birch (Betula), alder (Alnus), hazel (Corylus), hornbeam (Carpinus), olive (Olea), cedar (Cryptomeria and Juniperus), Plane tree (Platanus), the order of Poales including i.a. grasses of the genera Lolium, Phleum, Poa, Cynodon, Dactylis, Holcus, Phalaris, Secale, and Sorghum and the orders of Asterales and Urticales including i.a. herbs of the genera Ambrosia, Artemisia, and Parietaria. Other important inhalation allergens are those from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (superfamily Apidae), wasps (superfamily Vespidea), and ants (superfamily Formicoidae). Important inhalation allergens from fungi are i.a. such originating from the genera Alternaria, Cladosporium, Aspergillus and Penicillium.

Examples of food allergens are allergens from wheat, crustacean food including shrimp, prawn, crab and lobster, fish, hen's eggs, peanut, soy, cows' milk, nuts such as almond, brazil nut, cashew nut, hazelnut and walnut, celery, mustard and sesame seed.

Examples of recombinant allergens include but are not limited to proteins/peptides from plant pollens, grass pollens, tree pollens, weed pollens, insect venom, dust and storage mite proteins, animal dander, saliva, fungal spores and food allergens (i.e., peanut, milk, gluten and egg) prepared using recombinant techniques. Recombinant allergens can be obtained e.g. on a large scale by using microbial expression systems that may be grown on fermenters, produced by recombinant DNA techniques, or chemical precursors or other chemicals when synthesized chemically.

In one embodiment, the allergy is a food allergy. As used herein, an “food allergy” refers to a failure of oral tolerance to food antigens associated with Th2 immunity and allergen-specific IgE responses. That is, an immune response is generated in response to particular food antigens. Food intolerance often presents with symptoms similar to a food allergy, but does not involve an immune response. The most common food allergies include, but are not limited to, allergies to cow's milk, soy, wheat, eggs, dairy, peanuts, tree nuts, shellfish, fish, mushrooms, stone fruits, and other fruits. Food allergy affects an estimated 6 to 8 percent of children under age 3 and up to 3 percent of adults.

Common symptoms of food allergies include, but are not limited to, tingling or itching in the mouth; hives, itching or eczema; swelling of the lips, face, tongue and throat or other parts of the body; wheezing, nasal congestion or trouble breathing; abdominal pain, diarrhea, nausea or vomiting; dizziness, lightheadedness or fainting. During severe allergic reactions, anaphylaxis can occur, resulting in constriction and tightening of the airways; a swollen throat or the sensation of a lump in your throat that makes it difficult to breathe; shock with a severe drop in blood pressure; rapid pulse; and dizziness, lightheadedness or loss of consciousness. Untreated, anaphylaxis can cause a coma or even death.

In one embodiment, the food allergy is pollen-food allergy syndrome. Pollen-food allergy syndrome, also known as oral allergy syndrome, affects many people who have hay fever. In this condition, certain fresh fruits and vegetables or nuts and spices can trigger an allergic reaction that causes the mouth to tingle or itch. In serious cases, the reaction results in swelling of the throat or even anaphylaxis. Proteins in certain fruits, vegetables, nuts and spices cause the reaction because they're similar to allergy-causing proteins found in certain pollens. This is an example of cross-reactivity.

A food allergy can be exercised-induced food allergy. Eating certain foods may cause some people to feel itchy and lightheaded soon after starting to exercise. Serious cases may even involve hives or anaphylaxis.

In one embodiment, an anti-anaphylaxis agent is administered as a prophylactic treatment to a subject at risk of developing an allergy, e.g., a food allergy, that is capable of resulting in anaphylaxis. Risk factors for developing a food allergy include, but are not limited to a family history of asthma, eczema, hives, food allergy or other allergies; having other allergies, for example, to hay, pet dander, or seasonal allergies; a young age (e.g., newborn, infant, toddler, or child); and having asthma

Additional allergies capable of inducing anaphylaxis include, but are not limited to drug allergy, an insect allergy, a latex allergy, a mold allergy, a pet allergy, and a pollen allergy.

Additionally, provided herein is a method for treating or preventing the onset of anaphylaxis in a subject comprising administering an agent that inhibits RELMβ to a subject.

Further provided herein is a method for inducing tolerance to an allergen in a subject, comprising administering an agent that inhibits RELMβ to a subject. As used herein, the term “tolerance” refers to the process of suppressing a portion of the immune system that recognizes an antigen as being foreign. It will be appreciated by persons skilled in the art that the term “tolerance” as used herein has the same meaning as “immune tolerance”. As used herein, the expression “increasing tolerance” or “inducing tolerance” means an increase in tolerance to an antigen relative to the tolerance to the antigen prior to application of the method of the invention. In some embodiments, the term “tolerance” refers to the level of allergic response to a particular quantity of allergen. In some embodiments the tolerance can be oral tolerance and/or mucosal tolerance.

Provided herein is a method for reducing or eliminating a subject's immune reaction to an allergen, the method comprising administering an agent that inhibits RELMβ to a subject. In one embodiment, the subject's immune reaction to an allergen is reduced by at least 10%, at least 20%, at least 30%, at least 40%, at least 50%, at least 60%, at least 70%, at least 80%, at least 90%, at least 95%, at least 99% or more as compared to an appropriate control. As used herein, an “appropriate control” refers to the subject's immune reaction to an allergen prior to administration, or the immune reaction to an allergen in a subject who has been diagnosed as having an allergen. Methods for measuring an immune reaction to a food allergen can be performed by a skilled clinician, and include, but are not limited to, identifying IgE antibodies produced by a subject following exposure to a food allergen. To identifying if an immune response is reduced or eliminated, one skilled in the art can, e.g., measure the level of IgE antibodies produced by a subject prior to and following administration of the agent or fecal matter transplant, compare the levels, and identify a subject as having a reduced or eliminated immune response if the level of IgE after administration is lower than the level prior to administration.

In one embodiment, methods described herein further comprise, prior to administration, diagnosing a subject as having, or as being likely to develop, an allergy. In one embodiment, methods described herein further comprise, prior to administration, receiving the results of an assay that diagnoses a subject as having, or as being likely to develop, an allergy. Exemplary assays useful for diagnosing a subject as having or being at risk for having a food allergy are further described herein above.

In one embodiment, methods described herein further comprise, prior to administration, diagnosing a subject as having increased level of RELMβ as compared to the reference level. In one embodiment, methods described herein further comprise, prior to administration, receiving the results of an assay that diagnoses a subject as having increased level of RELMβ as compared to the reference level. As used herein, the “reference level” refers to the level of RELMβ in a healthy subject, e.g., not having an allergy. In one embodiment, the level of RELMβ is increased by at least 10%, at least 20%, at least 30%, at least 40%, at least 50%, at least 60%, at least 70%, at least 80%, at least 90%, at least 95%, at least 99% or more, or at least 2×, 3×, 4×, 5×, 6×, 7×, 8×, 9×, or more as compared to the reference level as compared to the reference level. One skilled in the art can assess the mRNA or protein level of RELMβ, e.g., in a biological sample, using PCR-based assays or Westernblotting, respectively.

### RELMβ

RELMβ belongs to the Resistin like molecule (RELM) family, a group of proteins that also includes RELM-α and RELM-γ and shares sequence homology to Resistin, an adipocyte-secreted factor. While RELMβ and Resistin were initially described as hormones that can regulate responsiveness to insulin [29], RELMβ has been subsequently found to be also involved in allergic inflammation and host defenses in the gut [30, 31]. RELMβ is expressed in intestinal epithelia cells and predominantly in colonic goblet cells [32]. RELMβ is induced by Th2 cytokines, as IL-4 and IL-13 can drive the differentiation of gut epithelial cells into RELMβ-producing goblet cells resulting in protection by worm infection [31]. RELMβ is upregulated during intestinal inflammation and plays a role in shaping the composition of the gut microbiota [33, 34]. RELMβ-KO mice show a higher proportion of organisms belonging to lactic acid bacterial species (such as L. gasseri and L. reuteri) and lower levels of Clostridia species (Clostridium coccoides) [35]. A recent study in a mucin-deficient mouse model of intestinal inflammation showed that RELMβ drives colitis by depleting commensal microbes and promoting dysbiosis [36]. Recent evidence further suggests that RELMβ promotes spatial segregation of the microbiota and colonic epithelium thus contributing to host-bacterial mutualism [37]. Finally, the inventors have observed that RELMβ-KO mice have marked decrease in intestinal mast cell load, indicating that RELMβ may be also involved in the regulation of mast cell responses in the gut.

Methods and compositions described herein require that the levels and/or activity of RELMβ are inhibited. As used herein, resistin-like beta precursor, also known as XCP2, FIZZ1, FIZZ2, HXCP2, RELMβ, RELMbeta, and RELM-beta, refers to a bactericidal protein that limits contact between Gram-negative bacteria and the colonic epithelial surface. RELMβ sequences are known for a number of species, e.g., human RELMβ (NCBI Gene ID: 84666) polypeptide (e.g., NCBI Ref Seq NP_115968.1) and mRNA (e.g., NCBI Ref Seq NM_032579.2). RELMβ can refer to human RELMβ, including naturally occurring variants, molecules, and alleles thereof. RELMβ refers to the mammalian RELMβ of, e.g., mouse, rat, rabbit, dog, cat, cow, horse, pig, and the like. The nucleic sequence of SEQ ID NO: 1 comprises a nucleic sequence which encodes RELMβ.

(SEQ ID NO: 1)

| | at |
|---|---|
| | ggggccgtcc tcttgcctcc |
| 121 | ttctcatcct aatccccctt ctccagctga tcaacccggg |
| | gagtactcag tgttccttag |
| 181 | actccgttat ggataagaag atcaaggatg ttctcaacag |
| | tctagagtac agtccctctc |
| 241 | ctataagcaa gaagctctcg tgtgctagtg tcaaaagcca |
| | aggcagaccg tcctcctgcc |
| 301 | ctgctgggat ggctgtcact ggctgtgctt gtggctatgg |
| | ctgtggttcg tgggatgttc |
| 361 | agctggaaac cacctgccac tgccagtgca gtgtggtgga |
| | ctggaccact gcccgctgct |
| 421 | gccacctgac ctga |

### RORγt-Expressing Regulatory T Cells

Methods described herein require that the population of RORγt-expressing regulatory T cells are increased following administering an agent described herein. Retinoic acid-related (RAR) orphan receptor gamma (RORγ) is a protein that in humans is encoded by the RORC (RAR-related orphan receptor C) gene. The RORC gene or the RORγt can also be referred to as RAR Related Orphan Receptor C, RAR-Related Orphan Receptor C, Nuclear Receptor Subfamily 1 Group F Member 3, Nuclear Receptor ROR-Gamma, Nuclear Receptor RZR-Gamma, NR1F3, RORG, RZRG, RAR-Related Orphan Nuclear Receptor Variant 2, Retinoid-Related Orphan Receptor Gamma, Retinoid-Related Orphan Receptor-Gamma, Retinoic Acid-Binding Receptor Gamma, RZR-GAMMA, IMD42, or TOR.

RORγt is produced from an mRNA identical to that of RORγ, with the exception that two 5′-most exons are replaced by an alternative exon, located downstream in the gene. The RORγt protein is a DNA-binding transcription factor and is a member of the NR1 subfamily of nuclear hormone receptors. RORγt is highly restricted to the thymus where it is expressed exclusively in immature CD4+/CD8+ thymocytes and in lymphoid tissue inducer (LTi) cells. RORγt plays an important regulatory role in thymopoiesis, by reducing apoptosis of thymocytes and promoting thymocyte differentiation into pro-inflammatory T helper 17 (Th17) cells. RORγt sequences are known for a number of species, e.g., human RORγt (NCBI Gene ID: 6097, SEQ ID NO: 3) and mouse RORγt (NCBI Gene ID: 19885, SEQ ID NO: 4) polypeptide (e.g., NCBI Reference Sequence: NP_001001523.1, SEQ ID NO: 5; NCBI Reference Sequence: NP_005051.2 and GenBank: AAH14804.1) and mRNA (e.g., NCBI Ref Sequence: NM_001001523.2, SEQ ID NO: 6; NCBI Ref Sequence: NM_005060.4). RORγt can refer to human RORγt, including naturally occurring variants, molecules, and alleles thereof. RORγt refers to the mammalian RORγt of, e.g., mouse, rat, rabbit, dog, cat, cow, horse, pig, and the like.

Regulatory T cells (also known as Tregs), also known as suppressor T cells, are a subpopulation of T cells that modulate the immune system, maintain tolerance to self-antigens, and prevent autoimmune disease. Tregs are immunosuppressive and generally suppress or downregulate induction and proliferation of effector T cells. Tregs can express the biomarkers CD4, FOXP3, and CD25 and are thought to be derived from the same lineage as naïve CD4 cells. Because effector T cells also express CD4 and CD25, Tregs can be very difficult to effectively discern from effector CD4+. The cytokine TGFβ promotes Tregs to differentiate from naïve CD4+ cells and is important in maintaining Treg homeostasis.

Methods for measuring the population of RORγt-expressing regulatory T cells are further described herein.

### Agents

In various embodiments, upon administration, an anti-anaphylaxis agent described herein increases the population of a RORγt-expressing regulatory T cells. In one embodiment, the population of RORγt⁺ regulatory T cells is increased by at least 50%, 60%, 70%, 80%, 90%, 95%, 99%, or more as compared to the population of RORγt⁺ regulatory T cells prior to administration, or the population of RORγt⁺ regulatory T cells of a subject having an allergy and/or anaphylaxis, or at risk of having an allergy and/or anaphylaxis.

In one aspect, an anti-anaphylaxis agent inhibits RELMβ is administered to a subject having, or at risk of having an allergy and/or anaphylaxis. In one embodiment, the agent that inhibits RELMβ is a small molecule, an antibody or antibody fragment, a peptide, an antisense oligonucleotide, a genome editing system, or an RNAi.

An agent is considered effective for inhibiting RELMβ if, for example, upon administration, it inhibits the presence, amount, activity and/or level of RELMβ in the cell.

In one embodiment, an agent that inhibits RELMβ increases the population of regulatory T cells that express RORγt. In one embodiment, the population of regulatory T cells that express RORγt is increased by at least 10%, by at least 20%, by at least 30%, by at least 40%, by at least 50%, by at least 60%, by at least 70%, by at least 80%, by at least 90%, by at least 99% or more as compared to an appropriate control. As used herein, an “appropriate control” refers to the population of regulatory T cells that express RORγt prior to administration of the agent or the population of regulatory T cells that express RORγt in a subject that is not administered the agent. One skilled in the art can determine if a population of regulatory T cells that express RORγt has been increased using standard techniques, for example, by identifying the population of regulatory T cells that express RORγt a cell sorting approach, e.g., FACS analysis or flow cytometry, via specific cell surface markers, and quantifying the size of the population, for example, by cell counts or population volume. Regulatory T cells that express RORγt can be readily identified, e.g., by the following cell surface markers: CD4, FOXP3, and CD25, and using an anti-RORγt antibody.

The Ikaros family member, Helios, has been reported as a marker to discriminate naturally occurring, thymic-derived Tregs from those peripherally induced from naïve CD4+ T cells. It was found that Helios-negative T cells are enriched for naïve T cell phenotypes and vice versa. Moreover, Helios can be induced during T cell activation and proliferation, but regresses in the same cells under resting conditions. In various embodiments, the regulatory T cell expressing RORγt has a lower expression, a higher expression, or the same expression of the Helios marker as compared to a regulatory T cell that does not express RORγt.

An agent can inhibit e.g., the transcription, or the translation of RELMβ. In one embodiment, mRNA and protein levels of RELMβ is reduced by at least 10%, by at least 20%, by at least 30%, by at least 40%, by at least 50%, by at least 60%, by at least 70%, by at least 80%, by at least 90%, by at least 99% or more as compared to an appropriate control. As used herein, an “appropriate control” refers to the mRNA and protein levels of RELMβ prior to administration of the agent or the mRNA and protein levels of RELMβ in a cell that is not contacted with the agent. To determine if an agent is effective at inhibiting RELMβ, mRNA and protein levels of RELMβ can be assessed using RT-PCR and western-blotting, respectively. Any known assays for measure a RELMβ's activity, for example determining if the population of RORγt-expressing Tregs are increased, which occurs when elevated levels of RELMβ are reduced.

An agent can inhibit the activity or alter the activity (e.g., such that the activity no longer occurs, or occurs at a reduced rate) of RELMβ in the cell (e.g., RELMβ's expression). In one embodiment, an agent that inhibits the activity of RELMβ by at least 10%, by at least 20%, by at least 30%, by at least 40%, by at least 50%, by at least 60%, by at least 70%, by at least 80%, by at least 90%, by at least 100% or more as compared to an appropriate control. As used herein, an “appropriate control” refers to the activity of RELMβ prior to administration of the agent, or the activity of RELMβ in a population of cells that was not in contact with the agent.

The agent may function directly in the form in which it is administered. Alternatively, the agent can be modified or utilized intracellularly to produce something which inhibits a RELMβ, such as introduction of a nucleic acid sequence into the cell and its transcription resulting in the production of the nucleic acid and/or protein inhibitor of the RELMβ. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In various embodiments, the agent is a small molecule that inhibits RELMβ. Methods for screening small molecules are known in the art and can be used to identify a small molecule that is efficient at, for example, increasing the population of RORγt-expressing Tregs, given the desired target, e.g., RELMβ.

In various embodiments, the agent that inhibits RELMβ is an antibody or antigen-binding fragment thereof, or an antibody reagent that is specific for RELMβ. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

In one embodiment, the agent that inhibits RELMβ is a humanized, monoclonal antibody or antigen-binding fragment thereof, or an antibody reagent. As used herein, “humanized” refers to antibodies from non-human species (e.g., mouse, rat, sheep, etc.) whose protein sequence has been modified such that it increases the similarities to antibody variants produce naturally in humans. In one embodiment, the humanized antibody is a humanized monoclonal antibody. In one embodiment, the humanized antibody is a humanized polyclonal antibody. In one embodiment, the humanized antibody is for therapeutic use.

In one embodiment, the antibody or antibody reagent in an anti-RELMβ antibody or antibody reagent and binds to an amino acid sequence that corresponds to the amino acid sequence encoding RELMβ (SEQ ID NO: 2)

(SEQ ID NO: 2)

| | |
|---|---|
| 1 | MGPSSCLLLI LIPLLQLINP GSTQCSLDSV MDKKIKDVLN |
| | SLEYSPSPIS KKLSCASVKS |
| 61 | QGRPSSCPAG MAVTGCACGY GCGSWDVQLE TTCHCQCSVV |
| | DWTTARCCHL T |

In another embodiment, the anti-RELMβ antibody or antibody reagent binds to an amino acid sequence that comprises the sequence of SEQ ID NO: 2; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 2. In one embodiment, the anti-RELMβ antibody or antibody reagent binds to an amino acid sequence that comprises the entire sequence of SEQ ID NO: 2. In another embodiment, the antibody or antibody reagent binds to an amino acid sequence that comprises a fragment of the sequence of SEQ ID NO: 2, wherein the fragment is sufficient to bind its target, e.g., RELMβ, and increases the population of RORγt-expressing Tregs.

In one embodiment, the agent that inhibits RELMβ is an inhibitory peptide. As used herein, an “inhibitory peptide” refers to a fragment polypeptide of a full length gene product, that when expressed in a cell, inhibits, e.g., the function, activity, and/or expression level of the full length gene product. For example, the inhibitory peptide can bind to a target of the full length gene product, preventing activation or silencing of that target by the full length gene product. An inhibitory peptide can comprise at least 5, at least 10, at least 15, at least 20, at least 25, at least 30, or more amino acids that are homologous to a portion of the amino acid sequence of RELMβ (SEQ ID NO: 2).

In one embodiment, the agent that inhibits RELMβ is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., RELMβ. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits RELMβ may comprise at least 5, at least 10, at least 15, at least 20, at least 25, at least 30, or more bases complementary to a portion of the coding sequence of the target, e.g., RELMβ.

In one embodiment, the antisense oligonucleotide that inhibits RELMβ may comprise at least 5, at least 10, at least 15, at least 20, at least 25, at least 30, or more bases complementary to a portion of the coding sequence the human RELMβ gene (e.g., SEQ ID NO: 1).

In one embodiment, RELMβ is depleted from the cell's genome using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment, the agent inhibits RELMβ by RNA inhibition. Inhibitors of the expression of a given gene can be an inhibitory nucleic acid. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of RELMβ. In some embodiments of any of the aspects, the agent is siRNA that inhibits RELMβ. In some embodiments of any of the aspects, the agent is shRNA that inhibits RELMβ.

One skilled in the art would be able to design siRNA, shRNA, or miRNA for inhibition of a target, e.g., using publically available design tools. siRNA, shRNA, or miRNA is commonly made using companies such as Dharmacon (Layfayette, Colo.) or Sigma Aldrich (St. Louis, Mo.).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference.

In one embodiment, the agent is miRNA that inhibits RELMβ. microRNAs are small non-coding RNAs with an average length of 22 nucleotides. These molecules act by binding to complementary sequences within mRNA molecules, usually in the 3′ untranslated (3′UTR) region, thereby promoting RELMβ mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

The agent may result in gene silencing of a target gene (e.g., RELMβ), such as with an RNAi molecule (e.g. siRNA or miRNA). This entails a decrease in the mRNA level in a cell for the target by at least about 5%, about 10%, about 20%, about 30%, about 40%, about 50%, about 60%, about 70%, about 80%, about 90%, about 95%, about 99%, about 100% of the mRNA level found in the cell without the presence of the agent. In one preferred embodiment, the mRNA levels are decreased by at least about 70%, about 80%, about 90%, about 95%, about 99%, about 100%. One skilled in the art will be able to readily assess whether the siRNA, shRNA, or miRNA effectively downregulates RELMβ, for example by transfecting the siRNA, shRNA, or miRNA into cells and detecting the levels of the mRNA or gene product found within the cell via PCR-based assays or western-blotting, respectively.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells, e.g., Treg cells, for example, that express RORγt. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

### Identifying a Subject at Risk

Certain aspects provided herein relate, in part, to the surprising finding that a subject having increased levels of RELMβ are at greater risk of having anaphylaxis. Provided herein is a method for identifying a subject at risk of having anaphylaxis comprising (a) obtaining a biological sample from a subject; (b) measuring the level of Resistin-like beta (RELMβ) in the biological sample of (a); and (c) comparing the level of (b) with a reference level, wherein a subject is identified as being at risk for anaphylaxis if the level of (b) is greater than a reference level.

In one embodiment, the subject has previously been diagnosed as having, or at risk of having being allergic to an allergen. In one embodiment, the subject has not previously been diagnosed as having, or is not at risk of having being allergic to an allergen.

In one embodiment, the level of RELMβ is greater by at least 10%, at least 20%, at least 30%, at least 40%, at least 50%, at least 60%, at least 70%, at least 80%, at least 90%, at least 95%, at least 99% or more, or at least 2-fold, 3-fold, 4-fold, 5-fold, 6-fold, 7-fold, 8-fold, 9-fold, or more as compared to the reference level as compared to the reference level. One skilled in the art can assess the mRNA or protein level of RELMβ, e.g., in a biological sample, using PCR-based assays or Westernblotting, respectively.

In one embodiment, the biological sample is a blood sample or tissue sample. For example, the biological sample is a peripheral blood sample, a sera sample, a tissue sample, a digestive tract tissue sample, a gastrointestinal tract tissue sample, a gut tissue sample, a stomach tissue sample, a small intestine tissue sample, or a large intestine tissue sample. In one embodiment, the biological sample is any sample that contains regulatory T cells. In one embodiment, the biological sample is taken from a subject that has previously been diagnosed with an allergy or anaphylaxis. In one embodiment, the biological sample is taken from a subject that has previously been diagnosed with and treated for an allergy or anaphylaxis. In one embodiment, the biological sample is taken from a subject that has not been diagnosed with an allergy or anaphylaxis. Methods for collecting samples from a subject are known in art and can be performed by a skilled person, e.g., via tissue biopsy or intravenous blood draw.

### Compositions

Any agent described herein, e.g., inhibitor of RELMβ, can be incorporated into compositions or pharmaceutical compositions suitable for administration to a subject for in vivo delivery to cells, tissues, or organs of the subject, or in vitro or ex vivo use thereof.

One aspect provides a composition comprising an agent that inhibits RELMβ, e.g., an anti-RELMβ antibody. In one embodiment, the composition further comprises a pharmaceutical carrier.

A further aspect provides a pharmaceutical composition comprising an agent that inhibits RELMβ. Typically, a pharmaceutical composition includes the agent or combination of agents described herein and a pharmaceutically acceptable carrier. For example, the agent or combination of agents can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high viral vector and antibiotic concentration. Sterile injectable solutions can be prepared by incorporating the agent or combination of agents in the required amount in an appropriate buffer with one or a combination of ingredients enumerated above, as required, followed by filtered sterilization. The composition can also include a pharmaceutically acceptable carrier.

Pharmaceutical compositions for therapeutic purposes typically must be sterile and stable under the conditions of manufacture and storage. The composition can be formulated as a solution, microemulsion, dispersion, liposomes, or other ordered structure suitable to high viral vector and antibiotic concentration. Sterile injectable solutions can be prepared by incorporating the viral vector and antibiotic in the required amount in an appropriate buffer with one or a combination of ingredients enumerated above, as required, followed by filtered sterilization.

As used herein, “pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation and is compatible with administration to a subject, for example a human. It must also be suitable for use in contact with any tissues or organs with which it may come in contact, meaning that it must not carry a risk of toxicity, irritation, allergic response, immunogenicity, or any other complication that excessively outweighs its therapeutic benefits. Examples of pharmaceutically acceptable carriers include, but are not limited to, a solvent or dispersing medium containing, for example, water, pH buffered solutions (e.g., phosphate buffered saline (PBS), HEPES, TES, MOPS, etc.), isotonic saline, Ringer's solution, polyol (for example, glycerol, propylene glycol, liquid polyethylene glycol, and the like), alginic acid, ethyl alcohol, and suitable mixtures thereof. In some embodiments, the pharmaceutically acceptable carrier can be a pH buffered solution (e.g. PBS) or water.

Compositions described herein can be formulated for any route of administration described herein below. Methods for formulating a composition for a desired administration are further discussed herein.

Compositions described herein can be used for prevention of anaphylaxis in subject having or at risk of developing an allergy and/or the treatment of anaphylaxis in subject having or at risk of developing an allergy. Further, compositions described herein can be used for inducing tolerance to an allergen in subject having or at risk of developing an allergy and/or reducing or eliminating a subject's immune reaction to an allergen.

### Administration

In some embodiments, the methods described herein relate to treating a subject having, diagnosed as having, at risk of having, or diagnosed as being at risk of having an allergen (e.g., a food allergy) or anaphylaxis, comprising administering an agent that inhibits RELMβ, or a microbiota therapeutic. Subjects having or at risk of having an allergy or anaphylaxis can be identified by a physician using current methods (i.e. assays) of diagnosing a condition. Symptoms and/or complications of allergen or anaphylaxis, which characterize these disease and aid in diagnosis are well known in the art and include but are not limited to, skin rash, digestive distress, constricted airway, inability to inflate/deflate lungs, or edema. Tests that may aid in a diagnosis of, e.g. an allergen, include but are not limited to skin tests that exposes the skin to concentrated amounts of a common food allergen, or blood tests. A family history of, e.g., an allergy or anaphylaxis, will also aid in determining if a subject is likely to have the condition or in making a diagnosis of an allergy or anaphylaxis.

The agents described herein can be administered to a subject having or diagnosed as having an allergy or anaphylaxis. The agents described can be administered to a subject at risk of having or diagnosed as being at risk of having an allergy or anaphylaxis. In some embodiments, the methods described herein comprise administering an effective amount of an agent to a subject in order to alleviate at least one symptom of, e.g., an allergy or anaphylaxis. As used herein, “alleviating at least one symptom of an allergy or anaphylaxis” is ameliorating any condition or symptom associated with, e.g., an allergy or anaphylaxis (e.g., skin rash, digestive distress, constricted airway, inability to fully inflate/deflate lungs, or edema). As compared with an equivalent untreated control, such reduction is by at least 5%, 10%, 20%, 40%, 50%, 60%, 80%, 90%, 95%, 99% or more as measured by any standard technique. A variety of means for administering the agents described herein to subjects are known to those of skill in the art. Such methods can include, but are not limited to oral, parenteral, intravenous, intramuscular, subcutaneous, transdermal, airway (aerosol), pulmonary, cutaneous, topical, or injection administration. In one embodiment, the agent is administered systemically or locally (e.g., to the lungs). In one embodiment, the agent is administered intravenously. In one embodiment, the agent is administered continuously, in intervals, or sporadically. The route of administration of the agent will be optimized for the type of agent being delivered (e.g., an antibody, a small molecule, an RNAi, a fecal transplant), and can be determined by a skilled practitioner. In some embodiments, the agent is administered orally, rectally, enterically, using a colonoscopy, using an enema, or using a plastic tube inserted through the nose into the gastrointestinal tract (e.g., stomach or intestines).

The term “effective amount” as used herein refers to the amount of an agent can be administered to a subject having, diagnosed as having, or at risk of having an allergy or anaphylaxis needed to alleviate at least one or more symptom of, e.g., an allergy or anaphylaxis. The term “therapeutically effective amount” therefore refers to an amount of an agent that is sufficient to provide, e.g., a particular anti-anaphylaxis effect when administered to a typical subject. An effective amount as used herein, in various contexts, would also include an amount of an agent sufficient to delay the development of a symptom of, alter the course of a symptom of, or reverse a symptom of an allergy or anaphylaxis. Thus, it is not generally practicable to specify an exact “effective amount”. However, for any given case, an appropriate “effective amount” can be determined by one of ordinary skill in the art using only routine experimentation.

In one embodiment, the agent is administered continuously (e.g., at constant levels over a period of time). Continuous administration of an agent can be achieved, e.g., by epidermal patches, continuous release formulations, or on-body injectors.

In one embodiment, the agent, is administered once every 2 weeks or once every 4 weeks. An agent described herein can be administered at least once a day, a week, every 2 weeks, every 3 weeks, a month, every 2 months, every 3 months, every 4 months, every 5 months, every 6 months, every 7 months, every 8 months, every 9 months, every 10 months, every 11 months, a year, or more.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring neurological function, or blood work, among others. The dosage can be determined by a physician and adjusted, as necessary, to suit observed effects of the treatment.

In some embodiments, the invention described herein relates to a pharmaceutical composition comprising an agent as described herein (e.g., an anti-anaphylaxis agent, such as an RELMβ inhibitor) as described herein, and optionally a pharmaceutically acceptable carrier. In some embodiments, the active ingredients of the pharmaceutical composition comprise an agent as described herein (e.g., RELMβ inhibitor). In some embodiments, the active ingredients of the pharmaceutical composition consist essentially of an agent as described herein. In some embodiments, the active ingredients of the pharmaceutical composition consist of an agent as described herein. In some embodiments, the carrier inhibits the degradation of the active agent described herein.

In certain embodiments, an effective dose of a composition comprising an agent as described herein can be administered to a patient once. In certain embodiments, an effective dose of a composition comprising an agent as described herein can be administered to a patient repeatedly. For systemic administration, subjects can be administered a therapeutic amount of a composition comprising an agent as described herein such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. an allergy or anaphylaxis, by at least 10%, at least 15%, at least 20%, at least 25%, at least 30%, at least 40%, at least 50%, at least 60%, at least 70%, at least 80% or at least 90% or more.

The dosing schedule can vary from once a week to daily depending on a number of clinical factors, such as the subject's sensitivity to an agent as described herein. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. An agent or composition described herein can be administered over a period of time, such as over a 5 minute, 10 minute, 15 minute, 20 minute, or 25 minute period.

### Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

The dosage should not be so large as to cause adverse side effects, such as immunosuppression or immunodeficiency. Generally, the dosage will vary with the age, condition, and sex of the patient and can be determined by one of skill in the art. The dosage can also be adjusted by the individual physician in the event of any complication.

In some embodiments, the methods described herein comprise administering an effective amount of an agent described herein to a subject in order to alleviate at least one symptom of, e.g., an allergy or anaphylaxis. In some embodiments, the dosage of the agent is approximately 100 mg per kilogram of the subject (mg/kg). In some embodiments, the dosage of the agent is 1 mg/kg-5 mg/kg, 5 mg/kg-10 mg/kg, 10 mg/kg-15 mg/kg, 15 mg/kg-20 mg/kg, 20 mg/kg-25 mg/kg, 25 mg/kg-30 mg/kg, 30 mg/kg-35 mg/kg, 35 mg/kg-40 mg/kg, 40 mg/kg-45 mg/kg, 45 mg/kg-50 mg/kg, 50 mg/kg-55 mg/kg, 55 mg/kg-60 mg/kg, 60 mg/kg-65 mg/kg, 65 mg/kg-70 mg/kg, 70 mg/kg-75 mg/kg, 75 mg/kg-80 mg/kg, 80 mg/kg-85 mg/kg, 85 mg/kg-90 mg/kg, 90 mg/kg-95 mg/kg, 95 mg/kg-100 mg/kg, 101 mg/kg-105 mg/kg, 105 mg/kg-110 mg/kg, 110 mg/kg-115 mg/kg, 115 mg/kg-120 mg/kg, 120 mg/kg-125 mg/kg, 125 mg/kg-130 mg/kg, 130 mg/kg-135 mg/kg, 135 mg/kg-140 mg/kg, 140 mg/kg-145 mg/kg, 145 mg/kg-150 mg/kg, 150 mg/kg-155 mg/kg, 155 mg/kg-160 mg/kg, 160 mg/kg-165 mg/kg, 165 mg/kg-170 mg/kg, 170 mg/kg-175 mg/kg, 175 mg/kg-180 mg/kg, 180 mg/kg-185 mg/kg, 185 mg/kg-190 mg/kg, 190 mg/kg-195 mg/kg, 195 mg/kg-200 mg/kg, 200 mg/kg-250 mg/kg, 250 mg/kg-300 mg/kg, 300 mg/kg-350 mg/kg, 350 mg/kg-400 mg/kg, 400 mg/kg-450 mg/kg, or 450 mg/kg-500 mg/kg.

### Combinational Therapy

In one embodiment, the agent described herein is used as a monotherapy. In one embodiment, the agents described herein can be used in combination with other known agents and therapies for treatment or prevention of an allergy or anaphylaxis. Administered “in combination,” as used herein, means that two (or more) different treatments are delivered to the subject during the course of the subject's affliction with the disorder, e.g., the two or more treatments are delivered after the subject has been diagnosed with the disorder or disease (for example, an allergy or anaphylaxis) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

Exemplary therapeutics used to treat or prevent an allergen, e.g., a food allergy, include, but are not limited to, Antihistamine, e.g., for the reduction or halting of an allergic reaction, Diphenhydramine (Benadryl, Banophen, Diphenhist, Wal-Dryl, and Nytol), Cetirizine (Zyrtec, Children's Cetirizine, Child Allergy Relf(cetirizine), All Day Allergy Relief(cetir), and Child's All Day Allergy(cetir)); Vasoconstrictor, e.g., for narrowing of blood vessels; Epinephrine (Adrenaclick, EpiPen, EpiPen Jr 2-Pak, Bronchial Mist Refill, and EPlsnap); and oral immunotherapy, e.g., Omalizumab (Xolair®).

When administered in combination, the agent and the additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment or prevention of a food allergy) is lower (e.g., at least 20%, at least 30%, at least 40%, or at least 50% lower) than the amount or dosage of each agent individually required to achieve the same therapeutic effect.

### Parenteral Dosage Forms

Parenteral dosage forms of an agents described herein can be administered to a subject by various routes, including, but not limited to, subcutaneous, intravenous (including bolus injection), intramuscular, and intra-arterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Controlled and Delayed Release Dosage Forms

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the agent as described herein can be administered in a sustained release formulation.

In some embodiments of the aspects described herein, an agent is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm & Haas, Spring House, Pa. USA).

### Efficacy

The efficacy of an agents described herein, e.g., for the treatment or prevention of a food allergy, can be determined by the skilled practitioner. However, a treatment is considered “effective treatment,” as the term is used herein, if one or more of the signs or symptoms of, e.g., an allergy or anaphylaxis, are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., decreased susceptibility to an allergy or anaphylaxis. Efficacy can also be measured by a failure of an individual to worsen as assessed by hospitalization, or need for medical interventions (i.e., progression and/or severity of an allergy or anaphylaxis). Methods of measuring these indicators are known to those of skill in the art and/or are described herein.

Treatment includes any treatment of a disease in an individual or an animal (some non-limiting examples include a human or an animal) and includes: (1) inhibiting the disease, e.g., preventing a worsening of symptoms; or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. increase of RORγt-expressing regulatory T cell). It is well within the ability of one skilled in the art to monitor efficacy of administration and/or treatment by measuring any one of such parameters, or any combination of parameters.

Efficacy can be assessed in animal models of a condition described herein, for example, a mouse model or an appropriate animal model of an allergen, e.g., food allergy, or anaphylaxis, as the case may be. When using an experimental animal model, efficacy of treatment is evidenced when a statistically significant change in a marker is observed, e.g., decreased susceptibility to a food allergen.

All patents, and other publications; including literature references, issued patents, published patent applications, and co-pending patent applications; cited throughout this application are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein or agent structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The invention can further be described in the following numbered paragraphs:
1) A method for identifying a subject at risk of having anaphylaxis, the method comprising:
   a. obtaining a biological sample from a subject;
   b. measuring the level of Resistin-like beta (RELMβ) in the biological sample of (a);
   c. comparing the level of (b) with a reference level, wherein a subject is identified as being at risk for anaphylaxis if the level of (b) is greater than a reference level; and optionally,
   d. administering to the subject identified as being at risk for anaphylaxis an anti-anaphylaxis therapeutic.
2) The method of any preceding paragraph, wherein the anti-anaphylaxis therapeutic is an agent that inhibits RELMβ.
3) The method of any preceding paragraph, wherein the anti-anaphylaxis therapeutic is a microbiota therapeutic.
4) The method of any preceding paragraph, further comprising, prior to obtaining the biological sample, diagnosing a subject as having, or likely to develop, an allergy.
5) The method of any preceding paragraph, further comprising, prior to obtaining the biological sample, receiving the results of an assay that diagnoses a subject as having, or likely to develop, an allergy.
6) The method of any preceding paragraph, wherein the subject is selected from the group consisting of: a newborn, an infant, a toddler, a child, and an adult.
7) The method of any preceding paragraph, wherein the allergy is a food allergy.
8) The method of any preceding paragraph, wherein the food allergy comprises at least one allergy to at least one food selected from the group consisting of: soy, wheat, eggs, dairy, peanuts, tree nuts, shellfish, fish, mushrooms, stone fruits, and other fruits.
9) The method of any preceding paragraph, wherein the agent is selected from the group consisting of: a small molecule, a compound, an antibody, a peptide, and an expression vector encoding an inhibitory nucleic acid or polypeptide.
10) The method of any preceding paragraph, wherein the antibody or antibody reagent is a humanized antibody or antibody reagent.
11) The method of any preceding paragraph, wherein the vector is non-integrative or integrative.
12) The method of any preceding paragraph, wherein the non-integrative vector is selected from the group consisting of an episomal vector, an EBNA1 vector, a minicircle vector, a non-integrative adenovirus, a non-integrative RNA, and a Sendai virus.
13) The method of any preceding paragraph, wherein the vector is a lentivirus vector.
14) The method of any preceding paragraph, wherein the agent increases the population of RORγt⁺ regulatory T cells.
15) The method of any preceding paragraph, wherein the agent reduces the level of RELMβ by at least 50%, 60%, 70%, 80%, 90%, 95% or more as compared to the level of RELMβ prior to administration.
16) The method of any preceding paragraph, wherein the expression of RELMβ is increased by at least 2×, 3×, 4×, 5×, 6×, 7×, 8×, 9×, or more as compared to the reference level.
17) The method of any preceding paragraph, wherein the population of RORγt⁺ regulatory T cells is increased by at least 50%, 60%, 70%, 80%, 90%, 95%, 99%, or more as compared to the population of RORγt⁺ regulatory T cells prior to administration.
18) The method of any preceding paragraph, wherein the reference level is the RELMβ level in a healthy patient.
19) The method of any preceding paragraph, wherein the microbiota therapeutic is a fecal matter transplant, wherein the fecal matter is obtained from a healthy subject.
20) The method of any preceding paragraph, wherein the biological sample is a sera or tissue sample.
21) A method for treating or preventing the onset of anaphylaxis in a subject, the method comprising: administering an agent that inhibits RELMβ to a subject.
22) A method for inducing tolerance to an allergen in a subject, the method comprising: administering an agent that inhibits RELMβ to a subject.
23) A method for reducing or eliminating a subject's immune reaction to an allergen, the method comprising: administering an agent that inhibits RELMβ to a subject.
24) The method of any preceding paragraph, further comprising, prior to administration, diagnosing a subject as having, or likely to develop, an allergy.
25) The method of any preceding paragraph, further comprising, prior to administration, receiving the results of an assay that diagnoses a subject as having, or likely to develop, an allergy.
26) The method of any preceding paragraph, further comprising, prior to administration, diagnosing a subject as having increased level of RELMβ as compared to the reference level.
27) The method of any preceding paragraph, further comprising, prior to administration, receiving the results of an assay that diagnoses a subject as having increased level of RELMβ as compared to the reference level.
28) A composition comprising an agent that inhibits RELMβ.
29) The composition of any preceding paragraph, wherein the agent is selected from the group consisting of: a small molecule, a compound, an antibody, a peptide, and an expression vector encoding an inhibitory nucleic acid or polypeptide.
30) The composition of any preceding paragraph, wherein the antibody or antibody reagent is a humanized antibody or antibody reagent.
31) The composition of any preceding paragraph, wherein the vector is non-integrative or integrative.
32) The composition of any preceding paragraph, wherein the non-integrative vector is selected from the group consisting of an episomal vector, an EBNA1 vector, a minicircle vector, a non-integrative adenovirus, a non-integrative RNA, and a Sendai virus.
33) The composition of any preceding paragraph, wherein the vector is a lentivirus vector.
34) The composition of any preceding paragraph, further comprising a pharmaceutically acceptable carrier.
35) A pharmaceutical composition comprising an agent that inhibits RELMβ.
36) Use of the composition of any preceding paragraph for the prevention of anaphylaxis in subject having or at risk of developing an allergy.
37) Use of the composition of any preceding paragraph for the treatment of anaphylaxis in subject having or at risk of developing an allergy.
38) Use of the composition of any preceding paragraph for inducing tolerance to an allergen in subject having or at risk of developing an allergy.
39) Use of the composition of any preceding paragraph for reducing or eliminating a subject's immune reaction to an allergen.
40) The use of any preceding paragraph, wherein the allergen is selected from the group consisting of: a food allergen, a drug allergen, an insect allergen, a latex allergen, a mold allergen, a pet allergen, and a pollen allergen.
41) A method for identifying a subject at risk of having anaphylaxis, the method comprising:
   a. obtaining a biological sample from a subject;
   b. measuring the level of Resistin-like beta (RELMβ) in the biological sample of (a); and
   c. comparing the level of (b) with a reference level,
42) wherein a subject is identified as being at risk for anaphylaxis if the level of (b) is greater than a reference level.

The technology described herein is further illustrated by the following examples which in no way should be construed as being further limiting.

### EXAMPLES

### Background

Atopic diseases are multifactorial conditions with environmental factors playing an increasingly important role. Allergic disorders are complex diseases that result from the interaction of multiple genetic, epigenetic and environmental influences [5]. Though allergic disorders are defined by their shared IgE mediated mechanisms, their markedly diverse clinical manifestations suggest that additional and unique factors are involved in shaping their heterogeneous phenotypical expression. The rapid increase in FA prevalence over the last few decades suggests a key contribution provided by environmental influences related to a modern lifestyle on genetically predisposed atopic individuals [13]. Several lifestyle factors have been identified that increase the risk of developing FA, including timing of exposure to food allergens [14], dietary habits [15] and microbial exposures [16-19]. The importance of timing of exposure to food allergens has been suggested by the failure of guidelines that delayed introduction of allergenic foods in children in halting the rise in FA [20] and confirmed by a seminal clinical trial of early peanut introduction which showed a marked decrease in the rate of peanut allergy in at-risk children fed peanut from infancy [21]. Among dietary factors, Vitamin D levels have been associated with increased risk of FA [22]. Finally, the observation that children raised and living on traditional farms have lower prevalence of allergies and asthma has suggested that colonization with certain microorganisms at mucosal surfaces can influence the developing immune system towards either tolerance or allergic inflammation (so called “hygiene hypothesis”) [16]. Indeed, multiple observational studies suggest that exposure to pet dogs [23], farm animals [24, 25], childcare attendance [18], older siblings in the family [18] and vaginal delivery [26] may protect against the development of atopic conditions, including FA. The concept that the microbiome plays a critical role in the development of atopic disease and FA is currently a focus of very active investigation. Overall these observations indicate an extensive network of environmental factors acting on predisposed individuals from early on in life. Understanding such contributions is key to achieve advances in the field of FA.

Cutaneous food exposure. The dual-allergen exposure theory postulates that exposure to food allergens through the skin can lead to allergic sensitization, while consumption of these foods at an early age leads to oral tolerance [27]. The presence of atopic dermatitis—with onset in the first months of life and requiring the use of topical steroids—is a strong risk factor for the development of FA [28]. Being able to identify those infants with atopic dermatitis that go on to develop FA possibly through cutaneous food exposure would allow to apply preventative strategies that can significantly decrease the development of FA in at risk individuals.

T regulatory cells play a crucial role in tolerogenic responses in the gut. T regulatory (Treg) cells play a central role in oral tolerance [38-42]. Impaired production and function of allergen-specific T regulatory (Treg) cells has been shown in FA [42]. The inventors have previously demonstrated that pathogenic Th2 reprogramming of Treg cells contributes to FA both in mice and humans [43]. The composition of the gut microbiome promotes the generation of mucosal ROR-γt⁺ Treg cells [44], which play a key role in suppressing FA and are decreased in FA in concomitance with the emergence of Th2 reprogrammed Treg cells [45].

Metabolomic profiling can provide key insights into complex disorders. High-throughput −omics technologies have emerged as powerful techniques to investigate complex diseases [46]. Situated downstream of the other −omics technologies, metabolomics can provide a snapshot of the physiological state of a system with the closest association to the phenotype (FIG. 1) [47]. The ability of metabolomic profiling to detect alterations that are driven both by endogenous and exogenous influences—including the microbiome [48, 49]—makes it particularly useful for unveiling pathways dysregulated as a result of both genetic and environmental factors and for biomarker discovery. Numerous studies over the last decade have identified metabolic signatures associated with asthma phenotype and severity [50]. The application of untargeted metabolomics to FA is so far limited. One study in a mouse model of peanut allergy showed alterations in uric acid metabolism, suggesting a possible role for alarmins—such as uric acid—in FA pathogenesis [51]. More recently, stool metabolomics suggested the possible involvement of sphingolipids in FA pathogenesis, mediated at least in part by modulation of iNKT cells [52]. The application of metabolomics to the study of FA holds the promise of contributing to the understanding of the integrated pathophysiological contributions in FA and of identifying biomarkers and metabolic pathways that can become the focus of targeted therapies.

The application of untargeted metabolomic profiling in a mouse model of FA has led to the identification of RELMβ as a potentially pathogenic cytokine not previously associated with FA, whose expression is dysregulated in mice and humans. Deletion of RELMβ in mice resulted in protection from FA reactions, with abolished mast cell responses and alterations in gut Treg cells profile. RELMβ-deficient mice display unique metabolomic signatures as compared to WT and FA prone mice. This application expands on these innovative approaches and observations to correlate RELMβ dysregulation in FA children with global, mast-cell and microbiome-associated metabolomic profiles as well as Treg cells mediated tolerogenic responses, with the goal to mechanistically relate pathognomonic changes in these profiles to alterations in RELMβ. The hypothesis that serum levels of RELMβ are uniquely elevated in subjects with FA as compared to those with other atopic conditions and correlate with pathognomonic metabolomic alterations, is an innovative hypothesis. Untargeted metabolomic profiling is a powerful tool for elucidating pathogenic mechanisms of disease. It has so far had limited application to FA, a condition in which it offers the potential of providing unbiased insight into factors shaping disease susceptibility and progression as well as leading to novel biomarkers of disease diagnosis and outcome that directly address currently unmet needs in FA. Relating changes in RELMβ with altered metabolomic profiles and imbalances in the Treg compartment can provide key mechanistic insights in the contribution of RELMβ to FA pathogenesis. Finally, and critically, the development of therapy for anaphylaxis based on the suppression of RELMβ over-expression in FA will provide a novel approach to anaphylaxis prevention in susceptible subjects by means of a long acting biologic therapy. Importantly, no such therapy currently exists.

Currently diagnostic modalities for FA have limited accuracy, and no disease-modifying treatment is available for FA patients, reflecting incomplete understanding of the complex pathogenic mechanisms underlying the development of FA and of the multiple environmental influences exerted on genetically predisposed atopic individuals. Experiments presented herein investigate the role of RELMβ as a FA biomarker and applies global and targeted metabolomic profiling to dissect pathways altered as a result of RELMβ dysregulation in FA, including the contribution of the microbiome and alterations in Treg-mediated gut tolerogenic responses. Elucidation of the mechanisms by which RELMβ contributes to FA pathogenesis may lead to the identification of candidate targets for future therapeutic intervention.

The care of patients with FA suffers from the limitations intrinsic to IgE-based testing, and further knowledge is clearly needed. Elucidating robust biomarkers of FA and underlying mechanisms linking the two may lead to new therapeutic options and intervention strategies for these children, or at least in specific FA disease endotypes such as the one characterized by RELMβ dysregulation.

### Approach

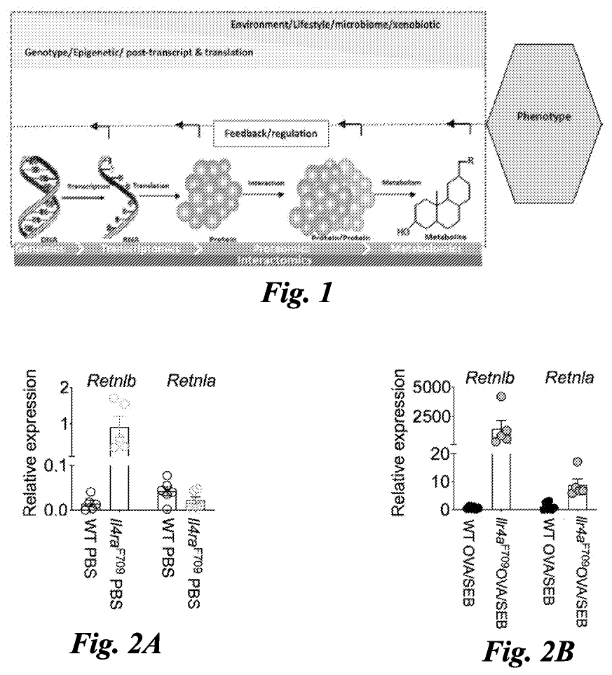

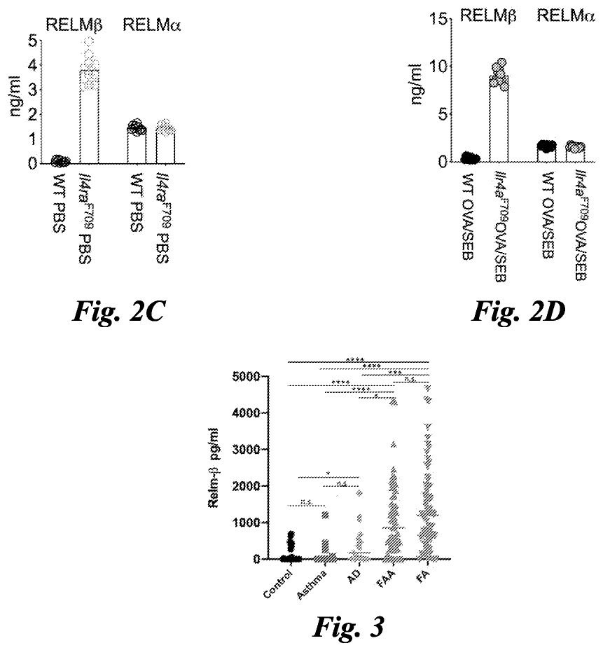

A unique mouse model of FA developed by the inventor which harbors a mutation in the immunotyrosine inhibitory motif (ITIM) of the IL-4 receptor α chain (Il4ra^{F709}) that potentiates receptor signaling is employed in experiments presented herein. Il4ra^{F709} mice exhibit robust oral sensitization to chicken egg Ovalbumin (OVA) and peanut, and develop severe IgE-dependent anaphylaxis upon oral allergen challenge [43, 54-56]. Untargeted serum metabolomic profiling of FA-prone Il4ra^{F709} mice and FA-resistant WT mice identified a marked dysregulation of lipid and carbohydrate metabolites (such as anhydroglucitol [57, 58]) in Il4ra^{F709} FA mice (data not shown). These results indicated that dysregulated lipid and sugar metabolisms in the context of Th2 inflammation are reflective of fundamental pathogenic mechanism(s) in FA and led to the evaluation of the role of members of the RELM family. The RELM family, composed of RELMα, RELMβ, and RELM-γ, is a group of proteins that share sequence homology to Resistin, an adipocyte-secreted factor that can regulate responsiveness to insulin [29]. Unlike Resistin, RELMα and -β are induced by Th2 cytokines [59]. RELMβ expression is particularly prominent in epithelial cells of the gastrointestinal tract, most notably goblet cells, where it has been implicated in parasite expulsion [31]. Previous studies by the inventors have shown increased Retnlb transcripts, encoding RELMβ, in Il4raF709 mice in the context of allergic inflammation [53]. Therefore, qPCR analysis in small intestinal tissue of Il4raF709 and WT mice was performed following either sham (PBS) or OVA sensitization, then oral challenge with OVA for all mice. Expression of Retnlb transcripts was observed to be increased in Il4raF709 relative to WT mice after sham sensitization, but was dramatically upregulated in the Il4raF709 mice following OVA sensitization and challenge (FIG. 2A). In contrast Retnla transcripts, encoding RELMα, were modestly increased (FIG. 2B). RELMβ protein concentrations were also measured in the sera of WT and Il4raF709 mice that were either sham (PBS) or OVA sensitized. The RELMβ serum concentrations were increased in the sham sensitized Il4raF709 mice and were further increased upon sensitization with OVA, whereas they were below the detection limit in WT controls. (FIGS. 2C and 2D). The Serum concentrations of RELMα were also analyzed in the same mouse groups. RELMα, has also been invoked in allergic airway inflammation and has also been associated with decreased glucose tolerance in experimental large intestinal inflammation [60]. However, and unlike RELMβ, the serum concentrations of RELMα were minimally changed between WT and Il4raF709 09 mice either before or after sensitization FIG. 2C, D). These results are consistent with divergent regulation of RELMβ versus RELMα in the context of FA

To determine whether human FA is associated with increased serum RELMβ levels, RELMβ concentrations in the sera of children with no known allergies (n=41), asthma (n=23), atopic dermatitis (AD) (17), asthma with FA (FAA) (55) and FA (n=66) were analyzed via ELISA The diagnosis of asthma was made based the Guidelines for the Diagnosis and Management of Asthma (EPR-3) criteria [61]. Subjects with isolated asthma did not suffer from eczema. Results revealed that RELMβ was highly increased in the overwhelming majority of subjects with FA and FAA as compared to asthmatics, subjects with atopic dermatitis and non-allergic controls (FIG. 3).

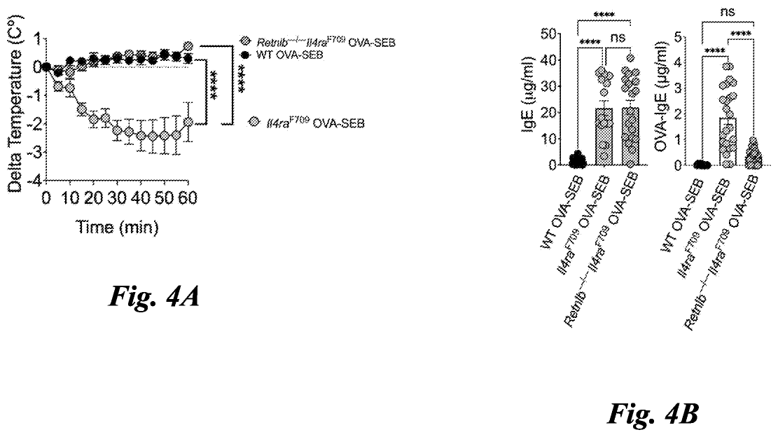

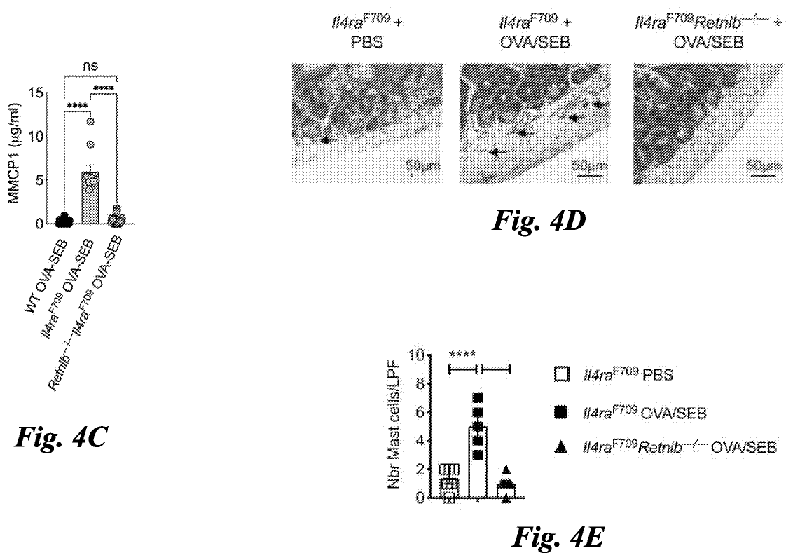

The sharp upregulation of RELMβ in FA-prone Il4raF709 mice and in human subjects with FA as compared to asthmatics and healthy control subjects is suggestive of a mechanistic function for RELMβ in disease pathogenesis. This hypothesis was validated by the observation that Il4raF709 Retnlb−/−—double mutant mice failed to develop FA in response to sensitization to OVA/SEB (FIG. 4A). While the OVA/SEB-sensitized Il4raF709 and Il4raF709 Retnlb−/−—double mutant mice showed a similar increase in their total serum IgE concentrations as compared to similarly sensitized WT mice, the Il4raF709 Retnlb−/−—double mutant mice were profoundly defective in their capacity to mount an allergen specific (OVA-specific) IgE response. not affect the increased total serum IgE concentrations in response A key effect of RELMβ deficiency appeared to be the suppression of mast cell release, as measured by the increased serum concentrations of the mast cell protease 1 (MMCP1) (FIG. 4C). The total number of mast cells, normally increased in OVA/SEB-sensitized Il4raF709 mice, was also suppressed in the similarly sensitized Il4raF709 Retnlb−/− double mutant mice (FIGS. 4D and 4E) or the Treg cell Th2 cell-like reprogramming (data not shown), otherwise cardinal steps in directing the FA response in Il4raF709 mice [45].

Investigating the relationship between RELMβ expression and the development and resolution of food allergy in children. Experiments are designed to (1) determine if expression of RELMβ is increased in young children at risk for FA and decreased following resolution of FA. It is specifically contemplated herein that increased RELMβ expression reflects pathogenic mechanisms that promote FA and is down-regulated upon acquisition of oral tolerance. (2) To compare RELMβ levels in young children with moderate to severe atopic dermatitis who are at risk for FA and children with mild AD, those with established FA or healthy controls. It is specifically contemplated herein that RELMβ elevation predicts those AD subjects at risk for the development of FA. (3) To compare RELMβ levels in children with active and resolved FA. It is specifically contemplated herein that RELMβ levels decline upon the induction of oral tolerance in FA subjects.

Rationale. RELMβ is upregulated in FA-prone Il4rd′ 709 mice and in human subjects with FA as compared to asthmatics and healthy control subjects. Mice deficient in RELMβ are protected from food allergen-induced anaphylactic reactions, indicating a mechanistic function for RELMβ in disease pathogenesis. In mice, elevation of RELMβ is observed at baseline, prior to sensitization, suggesting that it may serve as a marker of FA predisposition, prior to overt clinical manifestations elicited by food exposure. In a small group of children with AD and no FA, elevation of RELMβ was observed in a subset of subjects. Young children with AD are at increased risk of FA, with these two conditions often presenting as the first steps in the development of the atopic march, a typical progression of allergic disease that starts early in life with AD and FA and progresses to allergic rhinitis and asthma [62]. The inventors propose to investigate whether elevation of RELMβ is present in a subset of children with moderate to severe AD at higher risk of developing FA Such observation will be key in designing future prospective studies aimed at assessing the role of RELMβ as a candidate biomarker to predict FA

Experimental Design: Children ages 4 to 12 months with either mild or moderate/severe AD but no FA, infants with both eczema and FA, as well as healthy controls will be included. Infants with eczema but no FA will be monitored over 3 years for the development of FA by clinic follow-up visits to determine whether FA develops more frequently in children with elevated RELMβ. FA will be diagnosed based on the combination of both positive testing (specific IgE and/or skin testing) and a history of immediate symptoms occurring within 2 hours of ingestion of the culprit food. FA to foods that are avoided without a clear history of reaction, will be diagnosed by either a failed oral food challenge or serum specific IgE exceeding the established diagnostic cutoffs associated with 95% positive predictive values [7, 63, 64]. Children with positive testing and no history of reaction that do not fit the above criteria will be classified as non-FA, with secondary analyses performed looking separately at this group of sensitized children. Serum RELMβ levels will be measured by ELISA (Antigenix) at baseline as well as at follow-up visits to assess their relationship with eczema and FA over time. RELMβ levels will also be correlated with markers of FA and AD severity such total and specific IgE and SCORAD severity score. The presence of other atopic conditions (atopic dermatitis, asthma, allergic rhinitis) will be recorded and will be included either in adjusted or subgroups RELMβ analyses in order to adjust for the effect of other sources of Th2 inflammation and to test for possible interactions, for example between FA and asthma. Results will be validated in samples from a second cohort of patients with similar characteristics provided by Dr. Nadeau. Given the limited sample size of children with moderate/severe eczema and no FA, this study will serve as a pilot effort to determine the potential effect size and number of patients necessary for a larger prospective study analyzing the relationship between eczema, RELMβ and the development of FA.

Inclusion criteria. AD: clinical diagnosis with presence of eczematous, itchy and relapsing lesions with typical morphology and age-related distribution (facial, neck and extensor involvement in infants and children; flexural lesions at any age; sparing of the groin and axillary regions). AD severity will be assessed using the SCORAD severity scoring index (mild<25; moderate 25-50; severe>50). FA: documented FA phenotype, indicated by both of the following criteria: 1) A history of allergic reactions to one or more specific foods (e.g. milk, soy, egg, tree nuts, fish, shellfish, wheat or peanuts), including urticaria, angioedema, wheezing, anaphylaxis, diarrhea and/or vomiting, clearly triggered within 2 hours by food exposure and improving markedly after food avoidance. 2) Positive food specific skin test and/or serum specific IgE. Control subjects: Healthy subjects with no personal history of FA, asthma, eczema or allergic rhinitis. A sample size of 120 subjects (45 with severe moderate/severe AD, 25 with mild AD, 25 with FA and AD, and 25 controls) will provide 80% power to detect the hypothesized difference of 40% versus 10% RELMβ expression (alpha-0.05) in the moderate/severe AD group compared to mild eczema group and controls. N-45 with moderate/severe AD would provide 80% power to detect a significant difference of 30% developing FA vs. 5% developing FA in children with mild AD (probability at baseline=0.5), respectively. This assumes a 10% dropout rate from baseline to follow-tip.

It is specifically contemplated herein that a subset of children with moderate to severe AD will have elevated RELMβ levels. In this group of children RELMβ may serve as a marker of FA predisposition, reflecting the early establishment of pathogenic mechanisms in FA development, such as Th2 skewing, mast cell load, dysbiosis and/or others. Given the research limitations intrinsic to a career development award, follow-up will extend for 3 years, which may not be powered to fully inform on whether the infants with elevated RELMβ do go on to develop FA. Notwithstanding this limitation, this approach will allow us to test the hypothesis that children with more severe eczema may manifest increased blood RELMβ concentrations and a trend to develop FA. Results favoring this hypothesis will justify future studies employing prospective cohort(s) to further evaluate the use of RELMβ as a marker to predict FA in at-risk infants.

Rationale: Natural tolerance is acquired over time by a large proportion of children who develop FA early on in life, especially to allergens such as hen's egg and cow milk. It is not known whether such acquisition of tolerance involves extinction of pathogenic responses or development of alternative, protective immunological mechanisms. The inventors will test the hypothesis that the acquisition of oral tolerance is associated with the down-regulation of RELMβ expression by measuring the serum levels of RELMβ in children with active FA and children who have acquired tolerance to all previous allergenic foods.

Experimental Design: Serum samples of children with active FA (i.e. recent FA reaction) will be compared to those of children with history of FA who have acquired tolerance to all previously allergenic food(s). Analyses will be validated in a second cohort of patients with similar characteristics provided by Dr. Nadeau. FA diagnosis criteria are described herein above. Acquisition of food tolerance will be ascertained by the lack of response to an open food challenge performed as part of the clinical management. RELMβ levels will be compared between the two groups by ELISA. A sample size of 54 subjects (27 FA, 27 resolved FA) will provide 80% power to detect the hypothesized difference of SO % versus 20% RELMβ expression (alpha-0.05). Approximately 1,000 clinic oral food challenges are performed at BCH each year, assuring feasibility of this aim during the award timeline.

We anticipate that RELMβ levels are decreased in children who have outgrown their previous FA. It is possible that RELMβ may be decreased in all children with FA, in which case it may serve as a marker of acquisition of oral tolerance over time. Alternatively, it is possible that RELMβ levels may be decreased only in a subset of patients with resolved FA, while remaining persistently elevated in others. This latter observation may suggest that persistent RELMβ dysregulation is a marker of Th2 dysregulation that persists following resolution of FA in children who go on to develop other atopic manifestations. In this scenario RELMβ may serve as a marker of the atopic march. Finally, it is also possible that RELMβ may remain elevated in all children who have outgrown their FA, though this scenario appears unlikely. If RELMβ is indeed decreased in all or some of previously food allergic children, then prospective studies will be key in evaluating the temporal changes of RELMβ and its role in assessing the acquisition of oral tolerance.

Investigate metabolomic and immune regulatory mechanisms mediating RELM□ action in FA. Experiments are designed to (1) investigate whether increased RELMβ expression is associated with unique metabolomic signatures and changes in Treg cell tolerogenic responses in the gut. It is specifically contemplated herein that changes in RELMβ expression reflect pathogenic mechanisms in FA that result in altered metabolomic and Treg profiles and that may involve the gut microbiome. (2) To correlate global, mast-cell- and microbiome-related metabolomic profiles with RELMβ levels in food allergic children. It is specifically contemplated herein that unique metabolomic alterations correlate with changes in RELMβ expression in children with FA. (3) To correlate the frequencies of circulating ROR-γt⁺ and GATA3⁺ Treg cells with serum RELMβ levels and microbiome-derived metabolites in FA. It is specifically contemplated herein that RELMβ dysregulation modulates ROR-γt⁺ expression and alters balance of GATA3⁺:ROR-γt⁺ Treg cells by altering the production of microbiome-derived metabolites.

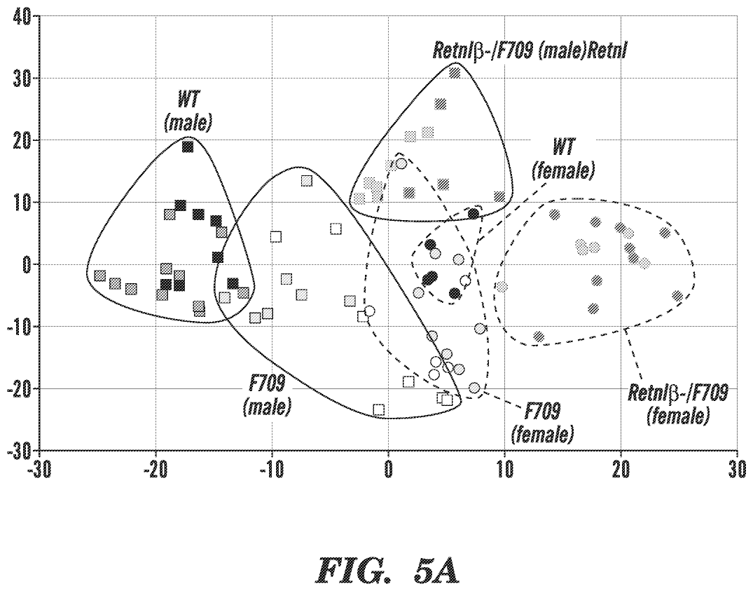

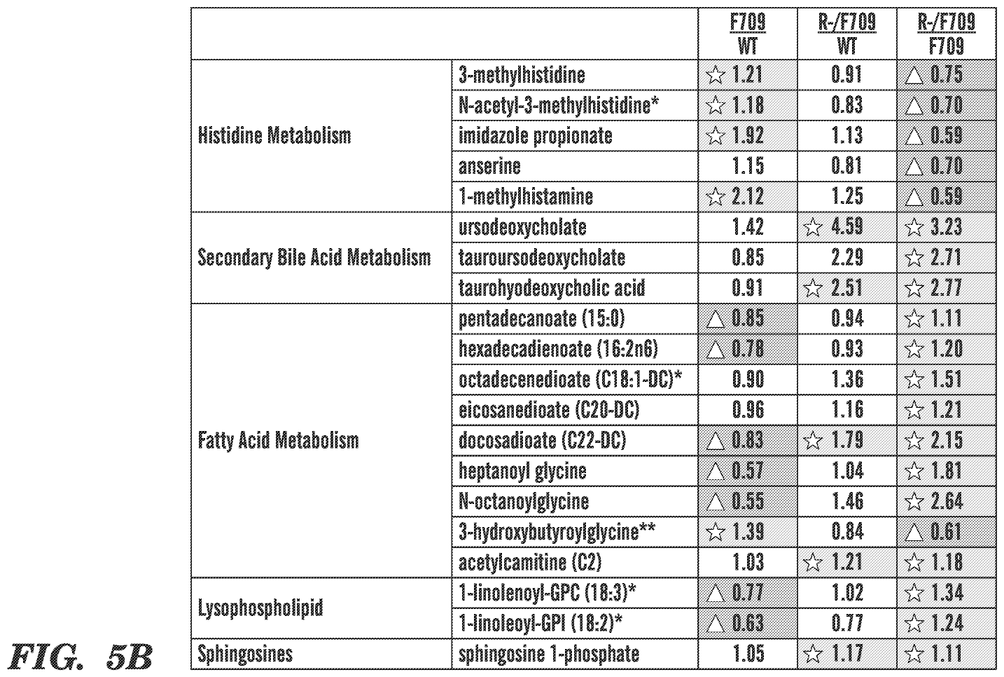

Rationale: RELMβ levels are elevated in a murine model of FA and in a large proportion of FA children, likely reflecting pathogenic mechanisms involved in FA. RELMβ deficient mice show a marked decrease in the mast cell load in the gut and in the release of mast cell mediators upon food challenge (FIGS. 4A-4E). Their metabolomic profile is distinct from that of WT and Il4ra^{F709} mice and is characterized by a decrease in histamine metabolites and alterations in lipids that are involved in mast cell function indicating that RELMβ may be a marker of mast cell load and identify individuals at risk for anaphylaxis. (FIGS. 5A-5B).

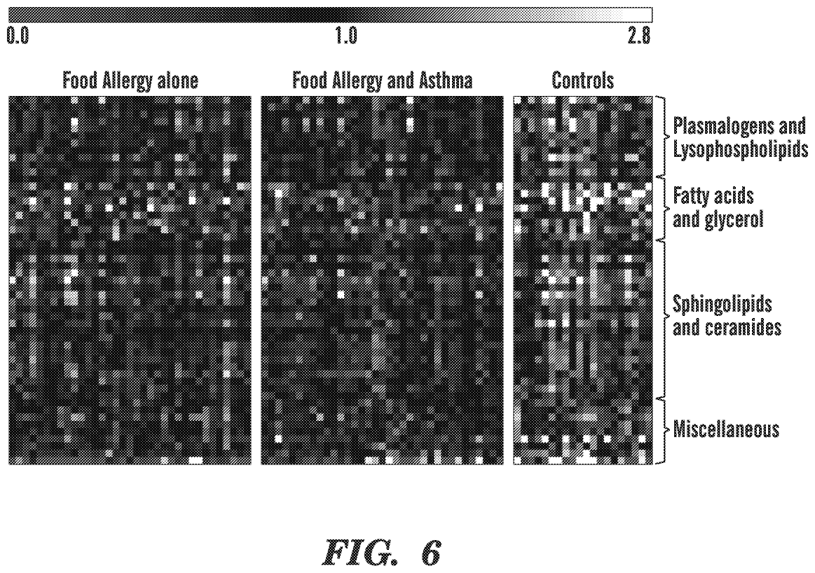

RELMβ has also been shown to modulate the murine microbiome through its bactericidal properties [36, 37]. RELMβ-deficient mice manifest an altered microbial composition [35] and their metabolomic profile shows alterations in microbiome sensitive metabolites such as fatty acids and secondary bile acids. The inventors have recently demonstrated in a pilot study that FA children display a unique metabolomic signature characterized by a prominent dysregulation in fatty acids, sphingolipid and plasmalogens (FIG. 6) and that severe FA phenotypes (i.e. history of multiple FA or anaphylaxis) are associated with changes in eicosanoids, fatty and in microbiome sensitive metabolites, including short chain fatty acids and aromatic amino acids (histidine and tryptophan) [65].

It is specifically contemplated herein that conserved metabolomic changes observed in mice and humans—including mast-cell and microbiome-associated signatures—correlate with the degree of RELMβ elevation in humans reflecting both systemic, mast cell and Treg-specific mechanisms involved in the pathogenesis and severity of FA.

Experimental Design: Untargeted (looking at general metabolism, including lipid and mast-cell related metabolites) and targeted (focusing on microbiome-related metabolites and short chain fatty acids) metabolomic profiling will be performed in an exploratory cohort of children with FA (n=50), asthma (n=35) and non-atopic controls (n=35), ages 1 to 11 years. RELMβ levels will be measured by ELISA. Metabolomic studies will be carried out at Metabolon, Inc. (Morrisville, N.C.) using a liquid-chromatography mass-spectrometry based approach. Plasma samples for metabolomics are processed and stored at −80 F within two hours of collection, according to recommended metabolomics practices. Exclusion criteria for metabolomic analyses include use of systemic steroids or immunosuppressive medications and use of antibiotics in the previous 6 weeks. Other medications and diet are recorded and included in the analyses. Bioinformatic tools (Metaboanalyst [66] and others) will be applied to identify individual metabolites (in the global and targeted panels) that segregate with FA and correlate with RELMβ levels among FA children. Metabolite enrichment analyses will be applied to gain insight into main altered pathways. Both unadjusted analyses and analyses adjusted for personal characteristics and atopic attributes will be performed. As seen in FIG. 3, increased RELMβ expression is present in the majority, though not all FA patients, defining a subgroup which manifests high serum levels of this cytokine, possibly consistent with intense Th2 cell skewing and high mast-cell load as seen in the Il4ra^{F709} mice. Accordingly, The inventor will perform a sub-analysis comparing the metabolomic profiles of FA children in the highest versus those in the lowest tertile of RELMβ expression, with the goal to dissect the FA endotype characterized by marked RELMβ dysregulation. History of anaphylaxis and other markers of disease severity will be compared between groups of differential RELMβ expression. Metabolite levels will also be correlated with personal characteristics (age, gender) disease attributes (type and number of FA, IgE levels etc) as well as presence of atopic comorbidities (allergic rhinitis, asthma, AD) in an attempt to gain insights into metabolomic pathways uniquely dysregulated in specific FA phenotypes. These investigations will be performed on samples already collected under the current IRB protocol #P00021163, which currently includes 60 FA, 40 asthma and 30 control plasma samples (ages<11 years). A second cohort of 50 FA patients and 30 controls will be recruited, who evaluates on average 10 FA and 5 asthma patients per week, and in the Asthma/Allergy Clinical Research Center at the Boston Children's Hospital and used for validation studies. Inclusion criteria for FA and controls are described in Aim 1a. Asthma diagnosis is based on EPR-3 criteria [61]. Metabolites associated with FA (q≤0.1) in the exploratory cohort will be the focus of targeted analyses in the validation cohort.

It is specifically contemplated herein that unique metabolomic alterations identified in FA children are reflective of pathogenic mechanisms related to RELMβ dysregulation. In particular, it is specifically contemplated herein that given the role of RELMβ in intestinal mast cell expansion and anaphylaxis in mice (FIGS. 4A-4E), the inventors will identify a subgroup of children with marked RELMβ elevation and increased lipid and mast-cell related metabolites (such as eicosanoid, PAF and histidine metabolites) who may be at risk of severe reactions due to augmented mast cell load and/or activation. Similarly, the inventors expect to observe alterations in microbial metabolites reflecting the impact of RELMβ on the gut microbiome composition. It is possible however that differences in mast-cell metabolites may be compounded by the presence of other atopic co-morbidities that are associated with heightened mast-cell responses in organ systems other than the gut. To address this possibility, the inventors will perform secondary analysis segregating patients by the presence of other atopic traits such as AD and allergic rhinitis. Furthermore, by correlating metabolites with personal and disease characteristics and comorbidities the inventors will be able to identify distinct metabolomic profiles in specific FA phenotypes that may suggest candidate pathogenic pathways and disease biomarkers both dependent and independent of RELMβ dysregulation

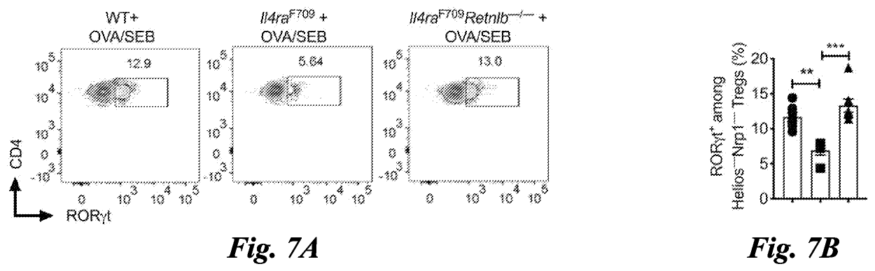

Rationale: Treg cells play a crucial role in the maintenance of tolerance to food allergens. In previous studies, the inventors have shown that Treg reprogramming cells towards a Th2 phenotype, characterized by increased expression of IL-4 and GATA3, is associated with FA both in mice and humans [43]. Th2 reprogramming of Treg cells is unaltered in RELMβ—deficient mice (FIG. 4E) suggesting that alternative mechanisms mediate RELMβ contribution to anaphylactic FA responses. It is appreciated that the microbiota regulates type 2 immunity through the induction of a specific subset of ROR-γt⁺ Treg cells [44]. Critically, the inventors have established that protection against FA is dependent on the induction by the microbiota of ROR-γt⁺ Treg cells [67]. Furthermore, while the microbiota of children with FA do not protect against FA when transferred into FA-prone germ free-mice, those of non-FA children do, in association with induction by the FA microbiota of ROR-γt⁺ Treg cells in the recipient mice [45]. In agreement with these findings, the inventors have observed a decreased proportion of circulating ROR-γt⁺ Treg cells in FA children [67]. The inventors have observed that Il4ra^{F709}Retnlb^{-/-} double mutant mice have increased proportions of ROR-γt⁺ Treg cells as compared to FA-prone Il4ra^{F709} mice, suggesting that RELMβ may contribute to FA pathogenesis by modulating microbiome-dependent gut ROR-γt⁺ Treg cell responses. (FIGS. 7A-7B).

A role for dysbiosis in the pathogenesis of FA has been suggested by mouse and human studies [45, 68]. In mouse models of FA, treatment with selected pro-tolerogenic bacterial species (Clostridiales, Bacteroidales) reverses peanut sensitization [45, 55]. In humans, several risk factors for FA (mode of birth, pet exposure, antibiotic treatment, presence of older siblings etc.) directly affect and modify the microbiome early in life. Furthermore, decreased microbiota diversity and unique microbial signatures in the infant gut—such as elevated ratio of Enterobacteriaceae/Bacteroidaceae—have been associated with the development of subsequent food sensitization [69]. These findings suggest that the pattern of early gut colonization can contribute to the development of atopic diseases, including FA [70]. RELMβ has been implicated in shaping the gut microbiome by depleting protective Lactobacillus species and by regulating the spatial segregation between the microbiota and the intestinal epithelium [36]. Notably, mice deficient in RELMβ have an altered microbiome as compared to WT mice [35]. The inventors postulate that RELMβ promotes dysbiosis and consequently changes in microbiome-derived metabolites that contribute to the FA response by modulating the frequency of ROR-γt⁺ Treg cells.

Experimental Design: FA children will be categorized based on serum RELMβ levels measured by ELISA (highest vs lowest tertile). Frequency of ROR-γt⁺ and GATA3⁺ expression will be measured by flowcytometry in CD25⁺ Foxp3⁺ Tregs isolated from PBMCs of children with FA (n=30), children with asthma (n=25), children with eczema (n=25) and non-atopic controls (n=20). The proportion of ROR-γt⁺ and GATA3⁺ Treg cells will be correlated with RELMβ levels in FA children. Proportions will also be compared in dichotomous comparisons of children with high vs. low RELMβ levels. Treg cell subpopulations will be measured also in children with asthma and non-atopic controls to determine the specificity of Treg imbalances to FA, as well in children with AD (with or without elevated RELM□) to determine whether they may be indicative of predisposition to FA development. The n=30 FA subjects will provide 80% power to detect a correlation of 0.63 between RELMβ expression and Treg profiles. Levels of microbiome-associated metabolites that are associated with increased RELMβ as described herein above will then be correlated with the frequency of ROR-γt⁺ Treg cells in FA children. This approach will allow to identify metabolites mediating the effect of RELMβ-induced dysbiosis on the Treg compartment.

It is specifically contemplated herein that the proportions of peripheral blood ROR-γt⁺ Treg cells in FA children inversely correlate with RELMβ levels and that microbiome-related metabolites can be identified that correlate with both RELMβ dysregulation and Treg imbalances. This would support the hypothesis that the contribution of RELMβ dysregulation in FA is at least in part mediated by changes in the gut microbial composition and that altered levels of microbial-derived metabolites in turn modulate Treg populations. The inventors anticipate identifying candidate metabolomic mediators of the effects of RELMβ in the gut that can act as biomarkers and target for therapeutic manipulation. In future studies will plan to address the direct effect of RELMβ on the human gut microbiome composition and the role of specific microbial species in the stool.

Generate and test in preclinical mouse models neutralizing monoclonal antibodies (mAbs) against human RELMβ. It is specifically contemplated herein that treatment of humanized mice with anti-human RELMβ mAbs protects against induction of FA-related anaphylaxis.

Rationale: The inventors' studies have demonstrated that RELMβ is essential for the development of anaphylaxis in mice, and that it is specifically elevated in FA subjects. It is specifically contemplated herein that neutralizing RELMβ in human subjects by specific mAb therapy would suppress the development of anaphylaxis and would promote long-term tolerance.

Experimental Design: Development of the RELMβ mAbs will be carried out in collaboration with the monoclonal antibody core (MAC) at the Dana Farber Cancer Institute (www.mabcore.dfci.harvard.edu).

Immunization Strategy: Two groups of 5 mice will be immunized with recombinant RELMβ protein (supplied by Peprotech) mixed with Freund's adjuvant using a standard immunization protocol. Group #1 will be standard mice (2-Balb/c; 2-057BL/6 and a Swiss-Webster) and group #2 will be all Notch 4 Knock-out mice. After 3 immunizations, the mice will be bled for titer determination 10 days following the last immunization. The MAC will run the titer by indirect ELISA using RELMβ protein and an irrelevant-Fc protein. Since the desired mAb should not cross-react with human Resistin, it will be necessary to titer them on recombinant Resistin as well (also obtained from Peprotech). Sera sample will be provided by the MAC to Dr. Chatila's lab for independent titer determination in a neutralizing assay. If the titer is sufficient, an animal will be selected for fusion. If the titer is insufficient, then the animals may be re-boosted to improve their titer. Unselected animals are maintained until the fusion screening process is completed (about a month from the fusion date).
Fusion Screening Strategy: Standard PEG-assisted hybridoma fusion using SP 2/0 myeloma cells and splenic cells from selected mouse. Eight plates are made per mouse unless the spleen has an unusually high or low number of cells, in which case the number of plates is adjusted to accommodate. After 10-14 days post-fusion, supernatants (120 ul/well) will be collected and available for screening The initial screening assay will be an indirect ELISA on RELMβ protein coated plates performed at the MAC. Positives (up to 24) will be expanded to 24 well plate and rescreened by indirect ELISA against irrelevant-Fc protein coated plates, also at the MAC. Additional wells may be selected for expansion off the fusion plates for additional charge. Supernatants (0.5 ml) will be made available for verification and additional characterization. This should include screening in a neutralizing assay. The MAC will expand up to 8 hybridomas to T25 flask and freeze 2 vials for back-up.
Subcloning Strategy: The MAC can subclone any of the selected hybridoma. It is strongly recommended that parental hybridomas be subcloned at least twice or until they have been shown to be clonal and stable. Up to 3 subclones may be selected for expansion from each parental subclone plate. Screening of the subclone plates will be done at the MAC. The MAC can re-subclone any selected subclone hybridomas at an additional cost per hybridoma per cloning, as specified on the quotation provided, until the hybridoma is stable. Final subclones selections will be isotyped. Two vials of each final selected subclone will be frozen. Once identified, selected mAbs can be humanized in preparation for preclinical and clinical trials. The MAC supports the humanization of mAbs using its own internal expertise and resources.
In vivo testing of blockade of human RELMβ protein in humanized mouse model of FA. To further test the activity of the neutralizing antibody, a humanized mouse model of FA will be used. For this, the SCID mouse (OD.Cg-Prkd^{csid} Il2rg^{tm1Wj1} Tg(CMV-IL3,CSF2,KITLG)1Eav/M^{loySzJ}) will be used [71]. These mice don't possess any active adaptive immune system. However, when engrafted with Cord blood-derived flow-sorted human CD34⁺ hematopoietic stem cells, they exhibit robust engraftment with functional human T and B lymphocytes and, importantly, human mast cells in their tissues, including the intestinal mucosa [71]. Following oral gavage feeding with peanut, they mount specific antibody responses, including peanut-specific IgE. Furthermore, when enterally challenged with PN, they exhibit mast cell mediated systemic anaphylaxis, as indicated by hypothermia and increases in plasma tryptase levels. Anti-IgE (omalizumab) treatment ablates this anaphylactic response.

The inventors propose to transfer CD34 cells from human donors to these mice to reconstitute their adaptive immune system with human immune system. The mice will be then sensitized with peanut flour then orally challenged with peanut, as described [71]. Subgroups of mice will receive either anti-RELMβ or isotype control mAbs at 100 μg intraperitoneal injection once weekly for the duration of the sensitization period, and the mice will be examined for their anaphylactic response as shown in FIGS. 4A-4E and the inventors' recent publications [43, 55, 67, 72, 73]. Other parameters examined will include total and peanut-specific IgE, MCPT1 release, tissue mastocytosis in the gut and especially the induction of RORγt⁺ Treg cells (and reciprocally, the suppression of Th2 cell-like reprogramed Treg cells and the recently described pathogenic Tfh13 [74, 75]) in the lamina propria of treated mice as a measure of restored immune tolerance.

In addition to the above prevention mode of therapy, the mice will undergo a curative mode as well. Mice will be orally sensitized with peanut to render them FA, then continued to be sensitized for an additional 4 weeks while receiving anti-RELMβ or isotype control mAbs at 100 μg intraperitoneal injection once weekly. The mice will then be challenged enterally with peanut flour and examined for their anaphylactic response.

It is specifically contemplated herein that the generated anti-human RELMβ mAbs would prove effective in both the prevention and curative modes of peanut FA therapy. Once the basic experiments are set, optimization as to the frequency and dosage of therapy in both prevention and curative mode. Of particular interest is the effect of anti-human RELMβ mAb therapy on the previously described dysbiosis in FA, as resetting the dysbiosis in favor of tolerance inducing (RORγt⁺ Treg cell inducing) bacteria, including Clostridiales and Bacteroidales species, will foster long-term oral tolerance [67]. These studies can be carried out in the inventors' laboratory using techniques readily available and described in the inventors' recent Nature Medicine publication [67].

### REFERENCES

1. Gupta, R., et al., The economic impact of childhood food allergy in the United States. JAMA Pediatr, 2013. 167(11): p. 1026-31.
2. Gupta, R. S., et al., The prevalence, severity, and distribution of childhood food allergy in the United States. Pediatrics, 2011. 128(1): p. e9-17.
3. Chafen, J. J., et al., Diagnosing and managing common food allergies: a systematic review. JAMA, 2010. 303(18): p. 1848-56.
4. Branum, A. M. and S. L. Lukacs, Food allergy among children in the United States. Pediatrics, 2009. 124(6): p. 1549-55.
5. Sicherer, S. H. and H. A. Sampson, Food allergy: Epidemiology, pathogenesis, diagnosis, and treatment. J Allergy Clin Immunol, 2014. 133(2): p. 291-307; quiz 308.
6. Sicherer, S. H. and H. A. Sampson, Food allergy. J Allergy Clin Immunol, 2010. 125(2 Suppl 2): p. S116-25.
7. Sampson, H. A., Utility of food-specific IgE concentrations in predicting symptomatic food allergy. J Allergy Clin Immunol, 2001. 107(5): p. 891-6.
8. Sampson, H. A. and D. G. Ho, Relationship between food-specific IgE concentrations and the risk of positive food challenges in children and adolescents. J Allergy Clin Immunol, 1997. 100(4): p. 444-51.
9. Lieberman, J. A. and S. H. Sicherer, Diagnosis of food allergy: epicutaneous skin tests, in vitro tests, and oral food challenge. Curr Allergy Asthma Rep, 2011. 11(1): p. 58-64.
10. Chokshi, N.Y. and S. H. Sicherer, Interpreting IgE sensitization tests in food allergy. Expert Rev Clin Immunol, 2016. 12(4): p. 389-403.
11. Patel, B. Y. and G. W. Volcheck, Food Allergy: Common Causes, Diagnosis, and Treatment. Mayo Clin Proc, 2015. 90(10): p. 1411-9.
12. Lamer, B. J., et al., Current Options for the Treatment of Food Allergy. Pediatr Clin North Am, 2015. 62(6): p. 1531-49.
13. Allen, K. J. and J. J. Koplin, Prospects for Prevention of Food Allergy. J Allergy Clin Immunol Pract, 2016. 4(2): p. 215-20.
14. Du Toit, G., et al., Early consumption of peanuts in infancy is associated with a low prevalence of peanut allergy. J Allergy Clin Immunol, 2008. 122(5): p. 984-91.
15. Grimshaw, K. E., et al., Diet and food allergy development during infancy: birth cohort study findings using prospective food diary data. J Allergy Clin Immunol, 2014. 133(2): p. 511-9.
16. Strachan, D. P., Hay fever, hygiene, and household size. BMJ, 1989. 299(6710): p. 1259-60.
17. Marrs, T., et al., Is there an association between microbial exposure and food allergy? A systematic review. Pediatr Allergy Immunol, 2013. 24(4): p. 311-320 e8.
18. Koplin, J. J., et al., Environmental and demographic risk factors for egg allergy in a population-based study of infants. Allergy, 2012. 67(11): p. 1415-22.
19. Hesselmar, B., et al., Pacifier cleaning practices and risk of allergy development. Pediatrics, 2013. 131(6): p. e1829-37.
20. Sicherer, S. H., et al., US prevalence of self-reported peanut, tree nut, and sesame allergy: 11-year follow-up. J Allergy Clin Immunol, 2010. 125(6): p. 1322-6.
21. Du Toit, G., et al., Randomized trial of peanut consumption in infants at risk for peanut allergy. N Engl J Med, 2015. 372(9): p. 803-13.
22. Allen, K. J., et al., Vitamin D insufficiency is associated with challenge-proven food allergy in infants. J Allergy Clin Immunol, 2013. 131(4): p. 1109-16, 1116 e1-6.
23. Gem, J. E., et al., Effects of dog ownership and genotype on immune development and atopy in infancy. J Allergy Clin Immunol, 2004. 113(2): p. 307-14.
24. Illi, S., et al., Early childhood infectious diseases and the development of asthma up to school age: a birth cohort study. BMJ, 2001. 322(7283): p. 390-5.
25. Braun-Fahrlander, C., et al., Environmental exposure to endotoxin and its relation to asthma in school-age children. N Engl J Med, 2002. 347(12): p. 869-77.
26. Laubereau, B., et al., Caesarean section and gastrointestinal symptoms, atopic dermatitis, and sensitisation during the first year of life. Arch Dis Child, 2004. 89(11): p. 993-7.
27. Lack, G., Update on risk factors for food allergy. J Allergy Clin Immunol, 2012. 129(5): p. 1187-97.
28. Martin, P. E., et al., Which infants with eczema are at risk of food allergy? Results from a population-based cohort. Clin Exp Allergy, 2015. 45(1): p. 255-64.
29. Rajala, M. W., et al., Adipose-derived resistin and gut-derived resistin-like molecule-beta selectively impair insulin action on glucose production. J Clin Invest, 2003. 111(2): p. 225-30.
30. Mishra, A., et al., Resistin-like molecule-beta is an allergen-induced cytokine with inflammatory and remodeling activity in the murine lung. Am J Physiol Lung Cell Mol Physiol, 2007. 293(2): p. L305-13.
31. Herbert, D. R., et al., Intestinal epithelial cell secretion of RELM-beta protects against gastrointestinal worm infection. J Exp Med, 2009. 206(13): p. 2947-57.
32. Artis, D., et al., RELMbeta/FIZZ2 is a goblet cell-specific immune-effector molecule in the gastrointestinal tract. Proc Natl Acad Sci USA, 2004. 101(37): p. 13596-600.
33. Hogan, S. P., et al., Resistin-like molecule beta regulates innate colonic function: barrier integrity and inflammation susceptibility. J Allergy Clin Immunol, 2006. 118(1): p. 257-68.
34. Nair, M. G., et al., Goblet cell-derived resistin-like molecule beta augments CD4+ T cell production of IFN-gamma and infection-induced intestinal inflammation. J Immunol, 2008. 181(7): p. 4709-15.
35. Okubo, H., et al., Involvement of resistin-like molecule beta in the development of methionine-choline deficient diet-induced non-alcoholic steatohepatitis in mice. Sci Rep, 2016. 6: p. 20157.
36. Morampudi, V., et al., The goblet cell-derived mediator RELM-beta drives spontaneous colitis in Muc2-deficient mice by promoting commensal microbial dysbiosis. Mucosal Immunol, 2016. 9(5): p. 1218-33.
37. Propheter, D. C., et al., Resistin-like molecule beta is a bactericidal protein that promotes spatial segregation of the microbiota and the colonic epithelium. Proc Natl Acad Sci USA, 2017. 114(42): p. 11027-11033.
38. Kim, K. S., et al., Dietary antigens limit mucosal immunity by inducing regulatory T cells in the small intestine. Science, 2016. 351(6275): p. 858-63.
39. Hadis, U., et al., Intestinal tolerance requires gut homing and expansion of FoxP3+ regulatory T cells in the lamina propria. Immunity, 2011. 34(2): p. 237-46.
40. Curotto de Lafaille, M. A., et al., Adaptive Foxp3+ regulatory T cell-dependent and -independent control of allergic inflammation. Immunity, 2008. 29(1): p. 114-26.
41. Cassani, B., et al., Gut-tropic T cells that express integrin alpha4beta7 and CCR9 are required for induction of oral immune tolerance in mice. Gastroenterology, 2011. 141(6): p. 2109-18.
42. Noval Rivas, M. and T. A. Chatila, Regulatory T cells in allergic diseases. J Allergy Clin Immunol, 2016. 138(3): p. 639-652.
43. Noval Rivas, M., et al., Regulatory T cell reprogramming toward a Th2-cell-like lineage impairs oral tolerance and promotes food allergy. Immunity, 2015. 42(3): p. 512-23.
44. Ohnmacht, C., et al., MUCOSAL IMMUNOLOGY. The microbiota regulates type 2 immunity through RORgammat(+) T cells. Science, 2015. 349(6251): p. 989-93.
45. Abdel-Gadir, A., et al., Microbiota therapy acts via a regulatory T cell MyD88/RORgammat pathway to suppress food allergy. Nat Med, 2019.
46. Patti, G. J., O. Yanes, and G. Siuzdak, Innovation: Metabolomics: the apogee of the omics trilogy. Nat Rev Mol Cell Biol, 2012. 13(4): p. 263-9.
47. Turi, K. N., et al., A review of metabolomics approaches and their application in identifying causal pathways of childhood asthma. J Allergy Clin Immunol, 2018. 141(4): p. 1191-1201.
48. Steinmeyer, S., et al., Microbiota metabolite regulation of host immune homeostasis: a mechanistic missing link. Curr Allergy Asthma Rep, 2015. 15(5): p. 24.
49. Heinken, A. and I. Thiele, Systems biology of host-microbe metabolomics. Wiley Interdiscip Rev Syst Biol Med, 2015. 7(4): p. 195-219.
50. Kelly, R. S., et al., Asthma Metabolomics and the Potential for Integrative Omics in Research and the Clinic. Chest, 2017. 151(2): p. 262-277.
51. Kong, J., et al., Comprehensive metabolomics identifies the alarmin uric acid as a critical signal for the induction of peanut allergy. Allergy, 2015. 70(5): p. 495-505.
52. Lee-Sarwar, K., et al., Intestinal microbial-derived sphingolipids are inversely associated with childhood food allergy. J Allergy Clin Immunol, 2018. 142(1): p. 335-338 e9.
53. Tachdjian, R., et al., In vivo regulation of the allergic response by the IL-4 receptor alpha chain immunoreceptor tyrosine-based inhibitory motif. J Allergy Clin Immunol, 2010. 125(5): p. 1128-1136 e8.
54. Mathias, C. B., et al., IgE-mediated systemic anaphylaxis and impaired tolerance to food antigens in mice with enhanced IL-4 receptor signaling. J Allergy Clin Immunol, 2011. 127(3): p. 795-805 e1-6.
55. Noval Rivas, M., et al., A microbiota signature associated with experimental food allergy promotes allergic sensitization and anaphylaxis. J Allergy Clin Immunol, 2013. 131(1): p. 201-12.
56. Burton, O. T., et al., Immunoglobulin E signal inhibition during allergen ingestion leads to reversal of established food allergy and induction of regulatory T cells. Immunity, 2014. 41(1): p. 141-51.
57. Akanuma, Y., et al., Urinary excretion of 1,5-anhydro-D-glucitol accompanying glucose excretion in diabetic patients. Diabetologia, 1988. 31(11): p. 831-5.
58. Yamanouchi, T., et al., Plasma 1,5-anhydro-D-glucitol as new clinical marker of glycemic control in NIDDM patients. Diabetes, 1989. 38(6): p. 723-9.
59. Nair, M. G., K. J. Guild, and D. Artis, Novel effector molecules in type 2 inflammation: lessons drawn from helminth infection and allergy. J Immunol, 2006. 177(3): p. 1393-9.
60. Munitz, A., et al., Resistin-like molecule alpha decreases glucose tolerance during intestinal inflammation. J Immunol, 2009. 182(4): p. 2357-63.
61. National Asthma, E. and P. Prevention, Expert Panel Report 3 (EPR-3): Guidelines for the Diagnosis and Management of Asthma-Summary Report 2007. J Allergy Clin Immunol, 2007. 120(5 Suppl): p. S94-138.
62. Hill, D. A. and J. M. Spergel, The atopic march: Critical evidence and clinical relevance. Ann Allergy Asthma Immunol, 2018. 120(2): p. 131-137.
63. Sicherer, S. H., E. H. Morrow, and H. A. Sampson, Dose-response in double-blind, placebo-controlled oral food challenges in children with atopic dermatitis. J Allergy Clin Immunol, 2000. 105(3): p. 582-6.
64. Perry, T. T., et al., Risk of oral food challenges. J Allergy Clin Immunol, 2004. 114(5): p. 1164-8.
65. Agus, A., J. Planchais, and H. Sokol, Gut Microbiota Regulation of Tryptophan Metabolism in Health and Disease. Cell Host Microbe, 2018. 23(6): p. 716-724.
66. Chong, J., et al., MetaboAnalyst 4.0: towards more transparent and integrative metabolomics analysis. Nucleic Acids Res, 2018. 46(W1): p. W486-W494.
67. Abdel-Gadir, A., et al., Microbiota therapy acts via a regulatory T cell MyD88/RORgammat pathway to suppress food allergy. Nat Med, 2019. 25(7): p. 1164-1174.
68. Rachid, R. and T. A. Chatila, The role of the gut microbiota in food allergy. Curr Opin Pediatr, 2016. 28(6): p. 748-753.
69. Azad, M. B., et al., Infant gut microbiota and food sensitization: associations in the first year of life. Clin Exp Allergy, 2015. 45(3): p. 632-43.
70. Savage, J. H., et al., A prospective microbiome-wide association study of food sensitization and food allergy in early childhood. Allergy, 2018. 73(1): p. 145-152.
71. Burton, O. T., et al., A humanized mouse model of anaphylactic peanut allergy. J Allergy Clin Immunol, 2017. 139(1): p. 314-322 e9.
72. Noval Rivas, M., et al., IL-4 production by group 2 innate lymphoid cells promotes food allergy by blocking regulatory T-cell function. J Allergy Clin Immunol, 2016. 138(3): p. 801-811 e9.
73. Burton, O. T., et al., Oral immunotherapy induces IgG antibodies that act through FcgammaRIIb to suppress IgE-mediated hypersensitivity. J Allergy Clin Immunol, 2014. 134(6): p. 1310-1317 e6.
74. Clement, R. L., et al., Follicular regulatory T cells control humoral and allergic immunity by restraining early B cell responses. Nat Immunol, 2019. 20(10): p. 1360-1371.
75. Gowthaman, U., et al., Identification of a T follicular helper cell subset that drives anaphylactic IgE. Science, 2019. 365(6456).

1. A method for identifying a subject at risk of having anaphylaxis, the method comprising:
a. obtaining a biological sample from a subject;
b. measuring the level of Resistin-like beta (RELMβ) in the biological sample of (a);
c. identifying a subject as being at risk for anaphylaxis if the level of (b) is greater than a reference level; and
d. administering to the subject identified as being at risk for anaphylaxis an anti-anaphylaxis therapeutic.

2. The method of claim 1, wherein the anti-anaphylaxis therapeutic is an agent that inhibits RELMβ.

3. The method of claim 1, wherein the anti-anaphylaxis therapeutic is a microbiota therapeutic.

4. The method of claim 1, further comprising, prior to obtaining the biological sample, diagnosing a subject as having, or likely to develop, an allergy.

5. The method of claim 1, further comprising, prior to obtaining the biological sample, receiving the results of an assay that diagnoses a subject as having, or likely to develop, an allergy.

6. The method of any of claims 1-5, wherein the subject is selected from the group consisting of: a newborn, an infant, a toddler, a child, and an adult.

7. The method of claim 4 or 5, wherein the allergy is a food allergy.

8. The method of any of claim 7, wherein the food allergy comprises at least one allergy to at least one food selected from the group consisting of: soy, wheat, eggs, dairy, peanuts, tree nuts, shellfish, fish, mushrooms, stone fruits, and other fruits.

9. The method of claim 2, wherein the agent is selected from the group consisting of: a small molecule, a compound, an antibody, a peptide, and an expression vector encoding an inhibitory nucleic acid or polypeptide.

10. The method of claim 9, wherein the antibody or antibody reagent is a humanized antibody or antibody reagent.

11. The method of claim 9, wherein the vector is non-integrative or integrative.

12. The method of claim 11, wherein the non-integrative vector is selected from the group consisting of an episomal vector, an EBNA1 vector, a minicircle vector, a non-integrative adenovirus, a non-integrative RNA, and a Sendai virus.

13. The method of claim 9, wherein the vector is a lentivirus vector.

14. The method of claim 2, wherein the agent increases the population of RORγt⁺ regulatory T cells.

15. The method of claim 2, wherein the agent reduces the level of RELMβ by at least 50%, 60%, 70%, 80%, 90%, 95% or more as compared to the level of RELMβ prior to administration.

16. The method of claim 1, wherein the expression of RELMβ is increased by at least 2×, 3×, 4×, 5×, 6×, 7×, 8×, 9×, or more as compared to the reference level.

17. The method of claim 14, wherein the population of RORγt⁺ regulatory T cells is increased by at least 50%, 60%, 70%, 80%, 90%, 95%, 99%, or more as compared to the population of RORγt⁺ regulatory T cells prior to administration.

18. The method of claim 1 or 16, wherein the reference level is the RELMβ level in a healthy patient.

19. The method of claim 3, wherein the microbiota therapeutic is a fecal matter transplant, wherein the fecal matter is obtained from a healthy subject.

20. The method of claim 1, wherein the biological sample is a sera or tissue sample.

21. A method for treating or preventing the onset of anaphylaxis in a subject, the method comprising: administering an agent that inhibits RELMβ to a subject.

22. A method for inducing tolerance to an allergen in a subject, the method comprising: administering an agent that inhibits RELMβ to a subject.

23. A method for reducing or eliminating a subject's immune reaction to an allergen, the method comprising: administering an agent that inhibits RELMβ to a subject.

24. The method of any of claims 21-23, further comprising, prior to administration, diagnosing a subject as having, or likely to develop, an allergy.

25. The method of any of claims 21-23, further comprising, prior to administration, receiving the results of an assay that diagnoses a subject as having, or likely to develop, an allergy.

26. The method of any of claims 21-23, further comprising, prior to administration, diagnosing a subject as having increased level of RELMβ as compared to the reference level.

27. The method of any of claims 21-23, further comprising, prior to administration, receiving the results of an assay that diagnoses a subject as having increased level of RELMβ as compared to the reference level.

28. A composition comprising an agent that inhibits RELMβ.

29. The composition of claim 28, wherein the agent is selected from the group consisting of: a small molecule, a compound, an antibody, a peptide, and an expression vector encoding an inhibitory nucleic acid or polypeptide.

30. The composition of claim 29, wherein the antibody or antibody reagent is a humanized antibody or antibody reagent.

31. The composition of claim 29, wherein the vector is non-integrative or integrative.

32. The composition of claim 31, wherein the non-integrative vector is selected from the group consisting of an episomal vector, an EBNA1 vector, a minicircle vector, a non-integrative adenovirus, a non-integrative RNA, and a Sendai virus.

33. The composition of claim 29, wherein the vector is a lentivirus vector.

34. The composition of claim 28, further comprising a pharmaceutically acceptable carrier.

35. A pharmaceutical composition comprising an agent that inhibits RELMβ.

36. Use of the composition of any of claims 28-35 for the prevention of anaphylaxis in subject having or at risk of developing an allergy.

37. Use of the composition of any of claims 28-35 for the treatment of anaphylaxis in subject having or at risk of developing an allergy.

38. Use of the composition of any of claims 28-35 for inducing tolerance to an allergen in subject having or at risk of developing an allergy.

39. Use of the composition of any of claims 28-35 for reducing or eliminating a subject's immune reaction to an allergen.

40. The use of claim 38 or 39, wherein the allergen is selected from the group consisting of: a food allergen, a drug allergen, an insect allergen, a latex allergen, a mold allergen, a pet allergen, and a pollen allergen.

41. A method for identifying a subject at risk of having anaphylaxis, the method comprising:
a. obtaining a biological sample from a subject;
b. measuring the level of Resistin-like beta (RELMβ) in the biological sample of (a); and
c. comparing the level of (b) with a reference level,
wherein a subject is identified as being at risk for anaphylaxis if the level of (b) is greater than a reference level.
